(19) United States
(12) Patent Application Publication
(10) Pub. No.: US 2020/0299702 A1
(43) Pub. Date: Sep. 24, 2020

(54) EXTRACELLULAR SECRETION OF TARGET PROTEIN

(71) Applicant: KOREA ADVANCED INSTITUTE OF SCIENCE AND TECHNOLOGY, Daejeon (KR)

(72) Inventors: Jung Hoon AHN, Busan (KR); Hyunjong BYUN, Daegu (KR); Jiyeon PARK, Busan (KR)

(21) Appl. No.: 16/811,024

(22) Filed: Mar. 6, 2020

Related U.S. Application Data
(63) Continuation-in-part of application No. PCT/KR2018/010466, filed on Sep. 7, 2018.

(30) Foreign Application Priority Data
Sep. 7, 2017 (KR) 10-2017-0114813
Mar. 19, 2018 (KR) 10-2018-0031579

(51) Int. Cl. C12N 15/70; C12N 15/78; C07K 14/21; C12N 9/20

(52) U.S. Cl. CPC C12N 15/70; C12N 15/78; C07K 2319/00; C12N 9/20; C12Y 301/01001; C07K 14/21

(57) ABSTRACT

The present invention provides a method for effective extracellular secretion of a target protein, by preparing a fusion protein connecting LARDS to the target protein and having pI lowered by adjusting the overall charge of target protein, and by using ABC transporter of a bacterial type 1 secretion system (T1SS). The method can allows a protein be produced at a large amount simply and effectively without a separate purification process.

### CROSS REFERENCE TO RELATED APPLICATIONS

This application claims the benefit of Korean Patent Application No. 10-2017-0114813 on Sep. 7, 2017 and Korean Patent Application No. 10-2018-0031579 on Mar. 19, 2018 with the Korean Intellectual Property Office, as well as PCT application No. PCT/KR2018/010466 on Sep. 7, 2018 with the WIPO, the disclosures of which are herein incorporated by reference in their entireties.

### TECHNICAL FIELD

The present invention relates to a method of performing or increasing secretion of a target protein linked to lipase ABC transporter recognition domain (LARDS), by using bacterial Type 1 Secretion system (T1SS) and lowering a pI, isoelectric point of the target protein in extracellular secretion of the target protein, and a method of producing a target protein efficiently.

### BACKGROUND ART

Mass production of recombinant proteins is an important issue in various industries. A general method for mass production of recombinant proteins is to synthesize recombinant proteins in prokaryotic cells such as Escherichia coli and then, lyse the cells and purify the cell extracts obtained by biochemical methods to produce recombinant proteins in a large scale.

Compared with the general method, a protein production system capable of simultaneously expressing and secreting recombinant protein in a cell is much more efficient and economical method since the need of expensive extraction and purification is reduced.

As the demand for protein products increases in clinical, industrial and academic fields, methods for efficient mass production of proteins from microorganisms have been developed. Some of the methods for mass production of proteins require that microorganisms produce target proteins in a culture medium and secret them extracellularly, in order to avoid the need for refolding proteins produced by manipulating microorganisms and purifying proteins intensively to isolate target proteins from proteins extracted from cells.

The desired proteins can be obtained without disruption of microorganisms, by engineering microorganisms to secret target proteins into a culture medium. Compared to current commercially available protein production systems using genetically engineered microorganisms, this method can minimize the contamination of protein products by intrinsic proteins of microorganisms due to no disruption of microorganism, thereby significantly reducing the cost of purification process.

### DISCLOSURE

### Technical Problem

The present invention provides a method of improving extracellular secretion of a target protein by regulating pI of a target protein recombined with LARDS and their whole charge, and a method of producing a target protein efficiently, by newly investigating a factor which determines secretion of a protein with a bacterial Type 1 Secretion system (T1SS).

### Technical Solution

The present inventors have found a method of mass production of a protein efficiently, and a new method of secretion and mass production of protein which can secret insoluble proteins extracellularly through bacterial T1SS (Type 1 Secretion system), as well as a method of mass production of a protein efficiently, and have completed the present invention. In addition, the present inventors have experimented to identify the differences between the proteins being capable of secretion and the proteins which are not secreted, among the proteins in which a lipase ABC transporter recognition domain (LARD3) is bound to a target protein to be secreted extracellularly.

Specifically, Pseudomonas fluorescens which mostly lives on the surface of plants has been consumed by humans for a long time, and therefore has been verified for its biological stability, and Pseudomonas fluorescens can endure various fermentation conditions under the high concentration of cell culture, and can produce a large amount of recombinant proteins. In addition, Pseudomonas fluorescens naturally has a number of secretion systems such as type I secretion system (T1SS) to type 6 secretion system (T6SS), and in particular, Pseudomonas fluorescens has the type 1 secretion system which transports heat-resistant lipase (TliA) through TliDEF of an ATP-binding cassette (ABC) carrier. Because of the applicability of Pseudomonas fluorescens for recombinant protein secretion, its transportation ability for some recombinant proteins has been proven and the secretion signal has been studied.

As the result of previous researches on protein mass production using P. fluorescens, it has been confirmed that for many proteins, when the recombinant proteins were prepared by conjugating lipase-ABC-transporter 3 (LARD3), the extracellular secretion was improved through TliDET transporter. However, most of some proteins were not secreted extracellularly and were present only in the cytoplasm, even though LARD3 was conjugated.

Accordingly, there is a need for researches on the identification of a factor determining whether a protein conjugated with LARD3 can be secreted by the ABC transporter, and a method of allowing extracellular secretion of a protein, which has not been increased even through the conjugation of LARD3, through the ABC transporter.

For the purpose, various protein genes bound to LARD3 were introduced to P. fluorescens, and the concentration of each protein was analyzed in supernatant and cell pellet of each culture. As a result, it has been confirmed that the pI of proteins has an important role in secretion using TliDEF that is the T1SS transporter of P. fluorescens, and it has been discovered that secretion of proteins with certain pI is promoted also in various T1SS transporters derived from microorganisms other than P. fluorescens, thereby completing the present invention.

Specifically, the present inventors used pDART plasmid vector developed in the previous research, to conveniently connect LARD3 to proteins (Ryu, J., Lee, U., Park, J., Yoo, D. H., and Ahn, J. H. (2015), A vector system for ABC transporter-mediated secretion and purification of recombinant proteins in Pseudomonas species. Appl Environ Microbiol 81, 1744-1753). The pDART plasmid has a multiple cloning site (MCS) directly followed by in-frame LARD3 gene, and the gene inserted to the multiple cloning site of pDART is expressed with LARD3 attached to its carboxyl terminus.

The LARD3 sequence is a sequence which is recognized by the ABC transporter of bacterial Type 1 Secretion System (T1SS) such as Pseudomonas fluorescens TliDEF, Pseudomonas aeruginosa AprDEF(PaAprDEF), Dickeya dadantii PrtDEF(DdPrtDEF), and Escherichia coli HlyBD+TolC, or the like, and which makes the recombinant proteins be secreted by the ABC transporter of T1SS.

In addition, pDART plasmid vector includes a Kanamycin-resistant gene for clone selection, has an origin of replication in broad host range to function as a shuttle vector between Escherichia coli and P. fluorescens, and comprises tliD, tliE, tliF genes expressing TliDEF complex.

Subsequently, the present inventors have attached an oligopeptide sequence to these proteins in order to artificially lower the pI value and add negative charge. To perform this work, the present inventors have produced two plasmids which attach aspartate polypeptide (oligo-aspartate) sequence to target proteins. After the experiment, the present inventors have produced a plasmid which attaches arginine polypeptide to target proteins, to investigate the effect when a positively charged amino acid is added to a target protein.

Lastly, the present inventors have experimented the secretion of supercharged mutants of the green fluorescent protein (GFP) developed in the previous research and have confirmed whether the supercharged mutants of the protein show a different secretion pattern from the original protein.

Type I secretion system (T1SS) means a polypeptide secretion system using an ABC transporter of bacteria, and is a chaperone-independent secretion system employing the Hly and TolC gene clusters. The secretion process is initiated by recognition of HlyA secretion signal and binding of HlyB to membrane. This signal sequence is very specific to the ABC transporter. Specifically, the HlyAB complex starts to untie coil by stimulating HlyD and TolC arrives at the outer membrane which recognizes terminal molecules or signals of HlyD. HlyD draws TolC to the inner membrane and HlyA is released outside of the outer membrane through a long-tunnel of protein channel.

The bacterial T1SS transports various molecules such as ions, drugs and proteins with various sizes (20 to 900 kDa). The secreted molecules have various sizes from small peptide colicimV (10 kDa) of Escherichia coli to cell adhesion protein (520 kDa) of Pseudomonas fluorescens. The proteins characterized well are RTX toxins and lipases. Type 1 secretion also involves in secretion of non-protein substrates such as cyclin β-glucans and polysaccharides.

T1SS is mainly present in Gram negative bacteria. Bacteria having T1SS includes Pseudomonas sp., Dickeva sp., E. coli, or the like, and more preferably, Pseudomonas fluorescens, Dickeya dadantii (or Erwinia chrysanthemi), Escherichia coli, Pseudomonas aeruginosa, or the like.

Because Pseudomonas fluorescens, Gram-negative bacterium does not accumulate acetic acid, it has resistance to the high cell concentration caused by fermentation conditions, and it is generally non-pathogenic to humans Thus, it is a candidate appropriate for protein production technology of proteins using secretion. In addition, Pseudomonas fluorescens is a Gram-negative psychrotrophic bacterium, and it has various excellent characteristics for recombinant protein production.

The present inventors have researched that a signal sequence recognized by an ABC transporter for polypeptide belonging to T1SS and a target protein are fused to secret the target protein into the culturing solution by ABC transporter of a microorganism, and as a result, have determined that the ABC transporters belonging to T1SS show the secretion efficiency of recombinant proteins proportionate to the isoelectric point (pI) of transport proteins, that is the charge property at pH 7. In other words, it has been confirmed that the negatively supercharged recombinant protein obtained by lowering pI of the target protein can increase the efficiency of secretion using the ABC transporter of T1SS, and pI has experimentally a close relation to the charge quantity of the protein (See FIG. 6).

Furthermore, the present invention has confirmed that the negatively supercharging has effect on the Type I secretion system other than TliDEF transporter of Pseudomonas fluorescens (See FIG. 19, FIG. 20, and FIG. 21). The T1SS means a polypeptide secretion system using an ABC transporter, and the TliDEF transporter is also a typical one of T1SS.

The present invention has isolated the genes of various T1SS transporters from microorganisms other than Pseudomonas fluorescens, specifically, three T1SS transporters of Pseudomonas aeruginosa AprDEF (PaAprDEF), Dickeya dadantii (also called Erwinia chrysanthemi) PrtDEF (DdPrtDEF), Escherichia coli HlyBD+TolC (E. coli expresses the original TolC protein). The three T1SS transporters have the amino acid sequence identity of 60%, 59% and 27% to that of TliDEF transporter of Pseudomonas fluorescens, respectively. It has been confirmed that the recombinant proteins to be secreted in which LARDS signal sequence is attached are secreted through the three transporters, respectively (See FIG. 19). Accordingly, it has been confirmed that the technology for improvement in the protein secretion by employing the negatively-supercharging is not only limited to the TliDEF transporter of Pseudomonas fluorescens, but also can be widely applied to T1SS transporters having the amino acid sequence identity of about 27% to TliDEF (See FIG. 20, FIG. 21, FIG. 22 and FIG. 23).

The present invention provides an expression vector for expression and extracellular secretion of a target protein in bacteria, comprising an expression cassette including a nucleotide sequence encoding a lipase ABC transporter recognition domain (LARD) and a nucleotide sequence encoding a target protein which are operably linked, where the LARD and target protein have acidic pI values and are expressed as fusion proteins secreted extracellularly.

According to an embodiment of the present invention, the expression vector may further comprise a nucleotide sequence encoding an ABC transporter of bacterial T1SS.

The term “target protein” means a target protein which can be produced at a large amount by producing biologically in bacteria and secreting extracellularly. The kind of the target protein is not particularly limited, and it may be cytokines, industrial enzymes, growth hormones, immune-related proteins, adhesive proteins, or the like. For example, it may be any one or more selected from the group consisting of Mannanase, MBP, NKC-TliA, Eg1V, GFP, thioredoxin, phospholipase A1, alkaline phosphatase, EGF, TliA, MAP, Capsid, Hsp40, M37 lipase, Cutinase, Chitinase, and CTP-TliA.

In order that pI of target protein can be adjusted to be an acidic pI of less than 7 by various methods. For example, the methods for adding acidic amino acids to the target protein, removing basic amino acids from the target protein, or substituting with other amino acid may be used, and the methods for supercharging a protein includes the manual supercharging comprising selecting amino acids which are amino acids protruding outside in the three-dimensional structure of the protein and do not affect the structure of protein, and substituting them with acidic amino acids, or the supercharging using Average Number of Neighboring Atoms Per Sidechain Atom (AvNAPSA) algorithm (1. Lawrence M S, Phillips K J, Liu D R. Supercharging Proteins Can Impart Unusual Resilience. Journal of the American Chemical Society 2007; 129: 10110-10112.). In this case, however, it is preferable to recombine so as not to affect the structure and the function of protein.

The target protein is a mutated protein with lowered pI value obtained by deleting at least one of the basic amino acids in the target protein, or by substituting them with other amino acids. The other amino acid is selected from the group consisting of acidic amino acids and neutral amino acids. At least one of the basic amino acids in the target protein is substituted with at least one amino acid selected from the group consisting of acidic amino acids and neutral amino acids.

The term “fusion protein” is a protein that is expressed in a connecting form of a nucleotide sequence encoding LARD and a nucleotide sequence encoding a target, and that has an acidic pI value and is secreted extracellularly.

According to an embodiment of the present invention, the pI value of the fusion protein may be less than 7, preferably 1 to 6, more preferably 2 to 5.5, or most preferably 3.0 to 5.5, for example 4.0 to 5.5.

When the pI value of the fusion protein is over 7, the amount of the protein secreted extracellularly through the ABC transporter of T1SS is significantly small in spite of the LARD-attached proteins, and most are present inside of cells. The protein having the pI value of the fusion protein less than 1 has very unstable structure, and therefore it is preferable to have a pI value in the range.

According to another embodiment of the present invention, the ABC transporter of T1SS may be a protein complex belonging to the T1SS transporter having the amino acid sequence identity of 20% or more, when the amino acid sequence identity is calculated to the parts of Pseudomonas fluorescens TliDEF according to the common calculation method for the nucleotide sequence identity (See FIG. 23). The ABC transporter of LipBCD of Serratia marcescens, specifically Serratia marcescens strain RSC-14 has 59% of amino acid sequence identity to TliD of Pseudomonas fluorescens, with 93% of Query coverage. According to an embodiment, the ABC transporter of T1SS having the amino acid sequence identity of 20% or more may be LipBCD of Serratia marcescens, HasDEF of Serratia marcescens, CyaBDE of Bordetella pertussis, CvaBA+TolC of Escherichia coli, RsaDEF of Caulobacter crescentus, Pseudomonas aeruginosa AprDEF (PaAprDEF), Dickeya dadantii PrtDEF (DdPrtDEF), Escherichia coli HlyBD+TolC or the like, but not limited thereto. Preferably, it may be Pseudomonas aeruginosa AprDEF (PaAprDEF), Dickeya dadantii PrtDEF (DdPrtDEF), and Escherichia coli HlyBD+TolC, and more preferably, it may be Pseudomonas fluorescens TliDEF.

The TliDEF are a multimer of three kinds of ATP binding cassette (ABC), membrane fusion proteins (MFP), and outer membrane proteins of TliD, TliE and TliF. The ABC protein selectively recognizes a secretion domain at the C-terminal part of a target protein and hydrolyzes ATP to secret the target protein. The membrane fusion proteins are embedded in cytoplasmic membrane and connect the ABC protein and outer membrane proteins. The outer membrane proteins are positioned in the outer membrane, and span most of cell periplasm forming channels through which the target protein is secreted.

In Pseudomonas fluorescens, the ABC protein, membrane fusion protein and outer membrane protein are encoded by tliD, tliE and tliF, respectively, which are located in the upstream of tliA in the lipase operon. The secretion/chaperone domain at the C-terminus of tliA is defined as a lipase ABC transporter recognition domain (LARD). Until now, five fragments of LARD with different lengths have been compared functionally, and it has been confirmed that LARD3 comprising 4 RTX (repeats-in-toxin) motifs is the most effective C-terminal signal in secretion using the ABC transporter. The Pseudomonas fluorescens including fusion protein construct of tliDEF and LARD3 can efficiently secret the LARD3-fused protein and obtain the secreted LARD3-fused protein directly from the culture broth.

According to one embodiment of the present invention, the nucleotide sequence encoding the target protein in the expression vector may further include a nucleotide sequence encoding an acidic peptide.

The number of amino acids in the added acidic peptides is not particularly limited, but according to one embodiment of the present invention, the number of amino acids comprised of the acidic peptide may be 6 to 20, preferably 7 to 15, for example, 10. When the number of amino acids comprised of the peptide is less than 6, the pH of the fusion protein does not show sufficient acidity, and thus may not be secreted efficiently through Type 1 secretion system (T1SS).

The acidic peptide may include one or more amino acids selected from the group consisting of aspartic acid and glutamic acid, and preferably, the nucleotide sequence encoding the acidic peptide may include a nucleotide sequence encoding the amino acid sequence of SEQ ID NO: 33 (10 aspartic acids; D10). The nucleotide sequence encoding the acidic peptide may be located at the 3′-terminus or 5′-terminus of the nucleotide sequence encoding the target protein, and preferably, it may be attached to the 3′-terminus.

In addition, the vector may further comprise a nucleotide sequence encoding a linker. The linker may be one to three peptides linked in which the each peptide consists of an amino acid sequence of Gly-Gly-Gly-Gly-Ser.

According to one embodiment of the present invention, the nucleotide sequence encoding the target protein may be obtained by removing one or more of basic amino acids contained in the target protein. The basic amino acid is lysine or arginine.

According to other one embodiment of the present invention, the target protein may be a supercharged target protein, or preferably negatively supercharged target protein. The present invention can increase the extracellular secretion of the target protein, by negatively supercharging the charge of the target protein which has not been secreted outside of Gram-negative bacterial cells before.

For example, a target protein may be negatively supercharged using a manual supercharging technique, which visualizes the three-dimensional structure of target proteins using software rendering, selects amino acids that are present relatively outside of proteins and have a functional group protruding on the direction of a solvent, so as not to largely affect the structure even though being changed, and then substituting them with aspartic acid and glutamic acid or substituting them with neutral amino acids, for example glutamine, when the amino acids are basic amino acids. As another example, the negatively supercharged target protein may be prepared by remodeling the protein surface using AvNAPSA (Average Neighbor Atoms per Sidechain Atom) algorithm. The protocol of AvNAPSA has been known well (WO2007/143574 A1). Specifically, the algorithm is algorithm digitizing and showing how much close each amino acid of proteins is to other atoms.

As shown in the examples, the present inventors have obtained a protein (negatively supercharged protein) sequence of which amino acids with 100 or less of AvNAPSA score (namely, amino acids which are present relatively outside of proteins and have a functional group protruding on the direction of a solvent, and therefore do not largely affect the structure even though being changed) are replaced with aspartic acid and glutamic acid according to the known protocol, have been synthesized to DNA sequence corresponding to the protein sequence and added the synthesized DNA sequence to pDART plasmid to express proteins for secretion. As a result, it has been confirmed that the efficiency of extracellular secretion of the negatively supercharged protein is significantly increased, compared to the proteins which are not negatively supercharged.

According to one embodiment of the present invention, the lipase ABC transporter recognition domain may be LARD1, LARD2 or LARD3. The LARD may mean a secretion/chaperon domain at the C-terminus of TliA in the heat-resistant lipase operon of Pseudomonas fluorescens. Specifically, the LARD peptide is classified into LARD1 to LARD 5 peptides by its sequence, and the LARD used in the present invention may be LARD 3, or preferably, LARD 3 peptide consisting of the amino acid sequence of SEQ ID NO: 22.

The LARD peptide including a sequence for purification being capable of functionally performing purification using hydrophobic chromatography, and the purification sequence is VLSFGADSVTLVGVGLGGLWSEGVLIS (SEQ ID NO: 29) which the present inventor discloses in Korean Patent No. KR10-1677090. The proteins including the purification sequence can be easily purified using hydrophobic interaction chromatography. Accordingly, the LARD3 peptide including the purification sequence may be used for purification of a target protein.

Furthermore, the LARD peptide includes a signal sequence of functionally inducing secretion from inside of cells to outside of cells, and the secretion signal sequence is GSDGNDLIQGGKGADFIEGGKGNDTIRDNSGHNTFLFSGHFGQD (SEQ ID NO: 30) which the present inventor discloses in Korean Patent No. KR10-1677090. The proteins including the secretion signal sequence may be secreted from inside of cells to outside of cells. Among LARD, LARD 1 to LARD 3 peptides including both the secretion signal sequence and the purification sequence, may be used for secretion to outside of cells and purification of the target protein. Preferably, the secretion signal sequence may be LARD 3 peptide (SEQ ID NO: 22).

The nucleotide sequence encoding the LARD is located at the 3′-terminus of the nucleotide sequence encoding a recombinant target protein, and it may encode a protein fused at the C-terminus of the recombinant target protein. When it is fused to the C-terminus of the recombinant target protein, the C-terminal signal sequence is not hydrolyzed advantageously, on the contrary to the signal sequence of the N-terminus hydrolyzed by extracellular secretion.

TABLE 1

| Name | Amino acid sequence | SEQ ID NO |
|---|---|---|
| LARD | VLSFGADSVT LVGVGL | 29 |
| purification | | |
| sequence | | |
| LARD secretion | GSDGNDLIQG GKGADFIEGG KGNDTIRDNS GHNTFLFSGH | 30 |
| signal sequence | FGQD | |
| LADR 1 | SIANLSTWVS HLPSAYGDGM TRVLESGFYE QMTRDSTIIV | 31 |
| | ANLSDPARAN TWVQDLNRNA EPHTGNTFII GSDGNDLIQG | |
| | GKGADFIEGG KGNDTIRDNS GHNTFLFSGH FGQDRIIGYQ | |
| | PTDRLVFQGA DGSTDLRDHA KAVGADTVLS FGADSVTLVG | |
| | VGLGGLWSEG VLIS | |
| LARD 2 | DSTIIVANLS DPARANTWVQ DLNRNAEPHT GNTFIIGSDG | 32 |
| | NDLIQGGKGA DFIEGGKGND TIRDNSGHNT FLFSGHFGQD | |
| | RIIGYQPTDR LVFQGADGST DLRDHAKAVG ADTVLSFGAD | |
| | SVTLVGVGLG GLWSEGVLIS | |
| LARD 3 | GSDGNDLIQG GKGADFIEGG KGNDTIRDNS GHNTFLFSGH | 22 |
| | FGQDRIIGYQ PTDRLVFQGA DGSTDLRDHA KAVGADTVLS | |
| | FGADSVTLVG VGLGGLWSEG VLIS | |

According to an embodiment of the present invention, a cell comprising the aforementioned expression vector is provided.

Specifically, the expression vector included in the cell may be an expression vector for expressing and secreting a target protein in bacteria, characterized by including an expression cassette in which a nucleotide sequence encoding a recombinant target protein and a nucleotide sequence encoding a lipase ABC transporter recognition domain (LARD) are operably linked, and expressing secretary proteins with an acidic pI value.

The cell may further comprise an expression vector including a nucleotide sequence encoding an ABC transporter of bacterial T1SS.

The cell may be a Gram-negative bacterium, and for example, may be Pseudomonas sp. strains, Dickeya sp., Escherichia sp., Xanthomonas sp., or Burkholderia sp. but not limited thereto. For example, in the present invention, the extracellular secretion of the target protein is achieved by functions of the ABC transporter. However, the ABC transporter functions in a Gram-negative bacterium with double membrane, and therefore, any Gram-negative bacterium may be used in the range of the present invention without limitation.

The Pseudomonas sp. may include any strain belonging to Pseudomonas sp., but for example, it may be Pseudomonas fluorescens, Pseudomonas fragi, Pseudomonas putida, Pseudomonas syringae, or Pseudomonas aeruginosa, and preferably, may be Pseudomonas fluorescens or Pseudomonas aeruginosa.

When the cell is Pseudomonas fluorescens, the target proteins introduced to Pseudomonas fluorescens may bind to the C-terminal signaling sequence of TliA and be secreted extracellularly in a form of fusion protein. The intrinsic lipase and protease of Pseudomonas fluorescens are also secreted extracellularly by the ABC transporter. Accordingly, when target proteins are expressed using wild type Pseudomonas fluorescens, or a strain producing complete lipase or protease, there is a problem that even though the target proteins are secreted extracellularly, the lipase and protease are mixed as impurities so as to make the next purification process become complex, and the protease hydrolyzes the produced target proteins.

Therefore, the Pseudomonas sp. may be a mutant of Pseudomonas fluorescens, in which some regions of one or more genes selected from the group consisting of lipase gene (tliA) and protease gene (prtA) of Pseudomonas fluorescens are deleted, and the partial deletion of genes are deleting gene regions so as to leave fragments with at least 100 bp or more of size at one or both of terminuses of the genes. The mutant strain may not produce one or more kinds of functional proteins selected from the group consisting of functional lipase and functional protease. The example of the mutant strain may be single deletion strain of lipase (mutant strain ΔtliA), single deletion strain of protease (mutant strain ΔprtA) and double deletion strain of lipase/protease (mutant strain ΔtliA ΔprtA). The contents of these mutant strains are described in detail in Korean patent publication 10-2004-0041159.

The mutant strains of Pseudomonas fluorescens do not produce functional protease protein, and may be achieved by all or partial deletion of the protease gene, or all or partial deletion of protease inhibitor gene (inh).

The mutant strain of Pseudomonas fluorescens may be, for example, the mutant strains of Pseudomonas fluorescens with accession number KCTC 12276BP, KCTC 12277BP, or KCTC12278BP, but not limited thereto.

According to an embodiment of the present invention, the present invention provides a method for producing target proteins in a cell, including preparing a cell transformed by the aforementioned expression vector, producing proteins to be secreted by culturing the cell, and separating or purifying the produced proteins.

The cell may further include an expression vector comprising a nucleotide sequence encoding an ABC transporter of bacterial T1SS.

The cell may be a gram negative bacterium. In the method for producing target proteins in a cell, the method for preparing a gram negative bacterial cell transformed by the expression vector may use general method of gene introduction, and for example, a recombinant vector in which a gene encoding target proteins is introduced may be introduced into the gram negative bacterium, or a gene encoding target proteins in the introduced vector may be inserted into genome by homologous recombination.

The vector may be all vectors including plasmid vector, cosmid vector, bacteriophage vector, virus vector and the like, but not limited thereto. The introduction of a vector into a gram-negative bacterium may be performed by the known methods such as electroporation, calcium phosphate (CaPO₄) precipitation, calcium chloride (CaCl₂) precipitation, PEG, dextran sulfate, lipofectamine, and the like.

In the culture of the cell, the culture conditions such as medium components, culturing temperature and culturing time, and the like may be appropriately controlled. Specifically, the culture medium may comprise all nutrients essential for growth and survival of microorganisms such as carbon source, nitrogen source, microelement components, and the like. In addition, the pH of the medium may be appropriately adjusted, and it may comprise components such as antibiotics. Moreover, expression of proteins may be induced by treating an inducer, and the kind of the treated inducer may be decided according to the vector system, and conditions such as inducer administration time and dosage and the like may be suitably controlled.

To effectively express target proteins in the wild type strain and mutant strain ΔtliA, 2× LB medium should be used. But, LB medium may be used for mutant strain ΔprtA and mutant strain ΔtliA ΔprtA, and the medium concentration may be decreased. In addition, the mutant strain ΔtliA ΔprtA has advantages that produce target proteins with the protection from PrtA hydrolysis without interruption of TliA outside of cells, can use LB medium and does not compete with lipase or protease-derived signal sequence, and thus, it makes production, secretion and purification of foreign proteins simple with the higher productivity, and thus it may be usefully used for mass production of target proteins.

The target proteins may be collected and purified by common methods, except for performing hydrophobic interaction chromatography using LARD including a purification sequence. For example, the cells collected by centrifugation may be disrupted using French press, ultrasonicator, and the like. When proteins are secreted to culture, the culture supernatant may be collected. When they are aggregated by overexpression, it may be obtained by dissolving proteins in an appropriate solution and denaturing and refolding. Oxidation and reduction systems of glutathione, dithiothreitol, β-mercaptoethanol, cystine and cystamine may be used, and a refolding agent such as urea, guanidine, arginine, and the like may be used, and some of salts may be used with the refolding agent.

As one embodiment, the method for producing target proteins in a gram negative bacterium may further comprise inserting a protease recognition site such as Factor Xa or Tobacco Etch Virus (TEV) protease, Enterokinase (EK), and the like, to cleave the acidic peptide and LARD bound to the target proteins, after isolating or purifying target proteins.

As one embodiment of the present invention, the purification of target proteins may use hydrophobic interaction. Then, the purification sequence of the present invention may be used as a purification tag. For example, the hydrophobic interaction chromatography is hydrophobic interaction chromatography using alkyl or aryl sepharose, and the alkyl group may be a methyl, ethyl, propyl or butyl group and aryl group may be phenyl group. Preferably, the alkyl group may be a methyl group. When a methyl group is used as the alkyl group, proteins comprising the purification sequence may be excellently purified, compared to using other alkyl groups.

Generally, for purification of proteins using a column, purification using His-tag are performed a lot, but the column for His-tag purification is costly, and also it is not appropriate for performing large capacity purification. In addition, NTA column is used a lot, but there is a problem in that Ni²⁺ or NTA is separated if reused, and there is a limit to repeated use. On the other hand, a hydrophobic column used in hydrophobic interaction chromatography has advantages in that it is a low-cost column and is highly economical and is suitable for using in large capacity separation, and has a high reuse rate.

Other embodiment of the present invention provides a method for performing or increasing extracellular secretion of proteins for secretion in a gram-negative bacterium, comprising inserting the aforementioned vector to a cell.

Specifically, method of performing an extracellular secretion of a target protein in a bacterial cell, comprising:

obtaining a target protein with lowered pI by deleting at least one basic amino acid in the target protein, or by substituting them with other amino acids,

preparing an expression cassette including a nucleotide sequence encoding Lipase ABC transporter recognition domain (LARD) and a nucleotide sequence encoding a target protein which are operably linked, wherein the LARD and the target protein have acidic pI and is expressed as a fusion protein, and

expressing the expression cassette in the bacterial cell.

In the method, at least one of the basic amino acids in the target protein is substituted with at least one amino acid selected from the group consisting of acidic amino acids and neutral amino acids. The other amino acids is at least one amino acids selected from the group consisting of aspartic acid, glutamic acid, and glutamine.

In an embodiment of the method, the target protein with lowered pI is negatively supercharged protein obtained by performing the following steps: selecting at least an amino acid not affecting the structure of target protein by having a functional group protruding in three dimensional structure of the target protein, and substituting the selected amino acid with at least one selected from the group consisting of acidic amino acids and neutral amino acids, when the selected amino acid is basic. Alternatively, the target protein is negatively supercharged or superneutralized protein obtained by performing the following steps: mutating at least one selected amino acid, into at least one selected from neutral amino acids and acidic amino acids to produce mutated target protein where the selected amino acids is carried out by selecting at least an amino acid not affecting the structure of target protein by having a functional group protruding in three dimensional structure of the target protein—and selecting the mutated target protein having activity. Specifically, the target protein is negatively supercharged protein obtained by performing the following steps: selecting at least an amino acid not affecting the structure of target protein by having a functional group protruding in three dimensional structure of the target protein, mutating at least one selected amino acid, into at least one selected from neutral amino acids and acidic amino acids to produce mutated target protein, and selecting the mutated target protein having activity.

The bacterial cell further comprises an ABC transporter of Type 1 Secretion System (T1SS), or an expression cassette comprising a nucleotide sequence encoding ABC transporter of bacterial Type 1 Secretion System (T1SS).

The present inventors have confirmed that the secretion rate of target proteins through the ABC transporter of bacterial Type 1 Secretion System (T1SS) is increased in negatively charged target proteins, and therefore, there is qualitative correlation between the pI of proteins and possibility of secretion by the T1SS ABC transporter.

In other words, proteins with strong acidic pI and strong negative charge are secreted by the ABC transporter of T1SS, but proteins with less negative charge and high pI are little secreted. As one embodiment, when genes are introduced to pFD10, secretion of some proteins is increased, and secretion of pBD10 is increased without reduction of expression.

According to the results of the pFD10 and pBD10 experiments and the comparison of secretion pattern of supercharged proteins performed in the following examples of the present invention, it has been confirmed that the secretion efficiency of proteins engineered for more negative charge is increased. The reason is why the energy required for overcoming membrane potential energy barrier between the cytoplasm and extracellular space is reduced.

Generally, the gram negative bacteria maintain the membrane potential of the inner membrane at about −150 mV and the cytoplasmic side is more negatively charged than the periplasm. This polarized charge distribution is maintained by various cell mechanisms including active proton transport across the membrane. The potential of the outer membrane also has a negative value, and the periplasm is charged more negatively than the extracellular space due to negatively charged membrane-derived oligosaccharide. However, due to many holes in the outer membrane, the magnitude of the outer membrane potential is commonly less than −30 mV.

Considering all these facts, secreting negatively charged proteins is generally advantageous in aspect of energy, and this affects equilibrium of the secretion reaction. The membrane potential is very powerful at the biochemical level, and has a significant effect on the change of free energy during transport through the ABC transporter. Transporting polypeptide across the inner membrane with −150 mV potential requires about 3.5 kcal/mol energy per charge which the polypeptide carries. The calculation at certain pressure, temperature and concentration is as follows.

w=−nFV=14.47 n kJ/mol=3.5 n kcal/mol

Herein, n is the total charge of polypeptide, and F is Faraday constant. In case of secreting proteins with ten positive net charge (N=+10), w=35 kcal/mol, and the secretion becomes that much worse. The typical value of the free energy change (AG) of ATP hydrolysis under the concentration of living body is 11.4 kcal/mol. The model suggested for the mechanism of the ABC transporter indicates that the ABC protein acts through continuous conversion between “inward” form and “outward” form associated with ATP hydrolysis. According to this model, one of the major power sources of the ABC transporter is power of “power stroke” that occurs in this process. The negatively charged membrane potential exerts electrostatic force on the charged polypeptide, promoting (for negative charges) or even decreasing (for the positive charges) for the force exerted by this power stroke, ultimately affecting secretion equilibrium.

The term, “membrane potential” hypothesis is supported by many previous researches across various secretion types. It has been reported that a positive-charge inducing mutation on E. coli lipoprotein interrupts protein folding near the membrane in both prokaryotic and eukaryotic organisms, thereby reducing secretion, and in addition, it has been discovered that the process of passing through the outer membrane was stopped, when the net negative charge of the passenger domain of E. coli autotransporter (type Va transport system) is neutralized or reversed.

In case of TliDEF, other factor to be considered is the state of charge of TliD. TliD is an ABC protein which is a component of the inner membrane of the TliDEF transporter. This protein has a nucleotide binding domain (NBD) and a transmembrane domain (TMD) which are linked by short sequence between domains. In particular, TliD ABC protein has a very high theoretical pI particularly, around the TMD (pI 9.43) and the sequence between domains (pI 8.14).

In homology-based structure of TliD, it has been shown that the dimer of this protein has positive charge distribution inside of the channel (FIG. 9A and FIG. 9B). This predicted model was prepared using Aquifex aeolicus PrtD (PDB ID 5122) with sequence homology of 40.98% as a template. In addition, the ConSurf homologue analysis on TliD has shown that this positive charge distribution in the central part of the channel is actually evolutionally conserved (FIG. 9C and FIG. 9D, yellow circle). Moreover, there is a positively charged residue which protrudes toward the opening of the window and blocks the substrate entry window in the ADP binding state of TliD, and the ConSurf results also verify that arginine or lysine is present at this position in all homologues (FIG. 9C, black arrow). The present inventors estimate that the inner surface of the positively charged channel promotes secretion by interaction with the negative-charged residue of the cargo protein during the protein transport (FIG. 9E).

In addition, the positive charge on the inner surface of the channel and substrate entry window may push the positive-charged section of polypeptide to prevent entry into the channel and ultimately block secretion (as could be seen from the results of the following experiments of attaching arginine polypeptide). Herein, the present inventors have hypothesized that proteins unfold (at least partially) during the transport process, and this is because the hole of TliF which is expected to have a very similar structure to E. coli TolC (1 tqq of PDB ID) has a mean inner diameter of 19.8 Å. This is clearly smaller than 20-30 Å, the mean diameter of most of sphere proteins including GFP barrel of 24 Å. TliF has a relatively rigid β-barrel form of transmembrane structure, and therefore is impossible to enlarge the hole during transport.

ABC transporters of other T1SS likewise have TMD whose ABC proteins are positively charged. HlyB-HlyD-TolC that is an E. coli haemolysin transport complex has significant positive charge distribution in TMD of ABC protein, HlyB, which is a homologue of TliD. Dickeya dadantii PrtD is also same. This fact intensively supports charge-dependence of T1SS ABC transporter secretion mechanism.

In conclusion, the present inventors have newly discovered that only highly acidic proteins can be transported through ABC transporters and basic or weak acidic proteins cannot be secreted through ABC transporters, and provides a method for improving secretion of target proteins extracellularly, by artificially lowering pI by attaching aspartic acid polypeptide to target proteins or negatively supercharging. In addition, a method is provided to confirm that an ABC transporter can secret the target protein through simple pI inspection, and ultimately, the range of proteins which can be efficiently produced through ABC transporter-dependent secretion can be extended by supercharging the proteins.

The present invention provides a method of practically modifying ABC transporter-incompatible proteins to be ABC transporter-compatible. These methods include the negative supercharging, superneutralization, random mutagenesis supercharging, and linear charge density-based supercharging.

The present invention verify hypotheses on the relationship between target protein's charged amino acid distribution and their secretion, and suggested that the substrate-charge related characteristics of ABC transporter-based secretion could be essentially same with that of the E. coli Sec transporters. In addition, the present invention provide a few methods of dealing with the non-secreted, or “incompatible” proteins, opening up a vast possibility of downstream applications for the ABC-transporters.

To be more specific, we hypothesized that if the charge density of any given region within the polypeptide string is lower (less positive and more negative) than a certain threshold, then the protein is “compatible” with the ABC transporter-mediated secretion, given that they are fused to a proper ABC transporter signal sequence. Potentially, there could be the following alternative hypothesis, based on the facts that both of the negative supercharging and selective superneutralizing, both of which improved secretion, remove positively charged amino acid residues from the target protein. The absence of positively charged amino acid residue is the critical determining factor of the ABC transporter-mediated secretion.

The present invention suggested a few methods to practically mutate the ABC-incompatible proteins. These methods were negative supercharging, selective superneutralization of positively charged residue and random mutation accompanied by activity screening. The present invention demonstrated that all of these methods can generate an ABC-compatible derivative of a given target protein. In addition, according to the results in FIGS. 20 to 22, the secretion-promoting effect of negatively supercharging the target proteins was also observed in other polypeptide-secreting T1SS ABC transporters. This suggests that the phenomena discussed in this study might potentially be more universal, rather than being specific to the P. fluorescens TliDEF ABC transporter complex.

Usage of mutagenesis approaches, such as supercharging technique, for secretion also has its own drawbacks. First, it is not a “one-for-all” type of solution. The change is performed on the cargo protein, not on the transporter complex. As a result, the manufacturer has to develop a supercharged version of the target protein each time a new target protein is introduced into the production line. This increases the development cost. In addition, the mutagenesis of the target protein always comes with the risk of losing the protein activity, especially when the level of mutagenesis is very high. In general, there is a “trade-off” relationship between the two properties in these kinds of situations. Introducing too many negative charges in pursuit of high secretion efficiency inevitably risks impairing the enzymatic activity of the target protein, damaging the profitability of the production scheme. Therefore, the developers are forced to undergo a heavy screening process, finding a “Goldilocks” variant that both possesses high secretion efficiency and retains the high activity. This is not an easy task, requiring extensive time and effort. To deal with this problem, we suggested superneutralization approach (more subtle charge change), variational mutation and screening approach (semi-random mutation and activity-based screening approach), and linear charge density analysis approach (minimizing the mutations), which might aid the developer in the course of optimizing the target protein for the efficient production in ABC-transporter based production line.

Bacterial ABC transporter's secretion dependence on the cargo protein charge status was studied in various aspects, revealing that the linear charge density of the cargo proteins might be the actual determinant of the ABC-dependent secretion, not the overall negative charge. We also provided very powerful solutions to make proteins compatible with ABC-transporter-mediated secretion and subsequent secretion-based production. Together, this characterizes the properties of ABC transporters and revolutionizes the potential of bacterial ABC transporters as the platform for efficient secretion-based protein production.

### Advantageous Effects

The present invention produces a protein for secretion with an acidic pI value, thereby providing a method of effectively secreting a target protein extracellularly through an ABC transporter of bacterial Type 1 Secretion System (T1SS). The method allows simple and efficient mass production of proteins without further purification processes.

### BRIEF DESCRIPTION OF THE DRAWINGS

FIG. 1a and FIG. 1b confirm the secretion of selected proteins according to Example 6, and represent the western blotting images showing expression and secretion of target proteins.

FIG. 2a and FIG. 2b show the correlation between the secretion ratio of the target proteins and their isoelectric points according to Example 6. The pI values of the target proteins are calculated for the sequence including attached LARD3.

FIG. 3a and FIG. 3b are results of expressing Lunasin and derivatives of Lunasin with different length of oligo-aspartic acid tail through LARD3 attachment and confirming secretion, in order to determine the optimal length of the oligo-aspartic acid sequence in P.fluorescens expression and secretion system according to Example 7.

FIG. 4 shows the structure of the plasmid used in the present invention according to Example 8, and shows the structure of pDART plasmid comprising MCS. Proteins fused with tliD, tliE, tliF and LARD3 are controlled by single operon. In case of (A), as there is the LARD3 gene right behind MCS, the inserted target gene is expressed with LARD3 attached to the C-terminus. (B) shows the structure of pFD10 which is a plasmid in which D10 sequence is attached to the N-terminus. The D10 gene directly follows start codon and is located right before the MCS and LARD3. (C) shows the structure of pBD10 plasmid, which attaches D10 sequence at the C-terminus, but before LARD3. The D10 gene is located between the MCS and LARD3.

FIG. 5 shows the result of detecting by western blotting and the lipase activity with a measurement medium, after adding 10 aspartic acids (D10) to the N-terminus (FD10) and C-terminus (BD10) of two kinds of TliA lipases (NKC-TliA, CTP-TliA) in which NKC and CTP sequences are attached, respectively, and expressing through pDART plasmid, according to Example 9.

FIG. 6 shows the result of western blotting detection, after adding 10 aspartic acids (D10) to the N-terminus (FD10) and C-terminus (BD10) of green fluorescent protein (GFP), mannanase, maltose binding protein (MBP) and Thioredoxin, and expressing through pDART plasmid, according to Example 10.

FIG. 7 shows the result of detecting with western blotting and lipase activity medium, after adding 10 aspartic acids (D10) or 10 arginines (R10), respectively, to the C-terminus of TliA lipase and green fluorescent protein (GFP) according to Example 11.

FIG. 8 shows the result of adding green fluorescent protein (GFP) supercharged by AvNAPSA method to pDART and expressing it, and detecting it by western blotting, according to Example 12.

FIG. 9 shows the charge distribution of TliD structure which is ABC protein of TliDEF complex according to Example 5. The parts indicated by circles in A, B, C of FIG. 9 show positively charged parts, and the parts indicated by the circle in D of FIG. 9 show pores inside of the transporter, and the white arrow inside of the circle represents the relatively negative atom and the black arrow represents the relatively positive atom.

FIG. 10a and FIG. 10b show the comparison of secretion of TliA, CTP-TliA and NKC-TliA according to Example 9, and FIG. 10a is the result of enzyme plate analysis of TliA, CTP-TliA and NKC-TliA, and FIG. 10b shows the western blotting result of TliA, CTP-TliA and NKC-TliA.

FIG. 11 shows the relation between the protein pI and charge at pH 7.0 according to Example 5.

FIG. 12 shows the result of predicting the structure of TliD according to Example 5.

FIG. 13 shows the result of prediction of transmembrane helices of modeled TliD according to Example 5. The rectangular box part corresponds to the transmembrane part predicted by the server.

FIG. 14 shows the result of ConSurf homologue conservation analysis of modeled TliD according to Example 5. The dark black parts are well conserved parts, and the lighter the color is, the less conserved it is.

FIG. 15 shows the protein secretion in pDAR-TliA, -NKC (-), NKC-L1, -NKC-L2, NKC-L3, -NKC-TliA according to Example 13. (A) Western blotting of TliA. (B) shows the result of enzyme plate analysis of TliA in different plasmids.

FIG. 16 shows the result of analysis of secretion of −10SAV, wtSAV, +13SAV and 2-10GST, wtGST, +19GST according to Example 14 (SAV: streptavidin/GST: glutathione 5-transferase).

FIG. 17 shows the result of inserting and expressing glutathione S-transferase (GST) supercharged by replacing protruding amino acids with aspartic acid or arginine and streptavidin (SAv) to pDART, and detecting with western blotting, while looking at the structure without using AvNAPSA (Average Number of Neighboring Atoms Per Sidechain Atom) method according to Example 14.

FIG. 18 shows the result of highly negatively charging MelC₂ tyrosinase, cutinase (Cuti), chitinase (Chi) and M37 lipase by AvNAPSA method, and then adding highly negatively charged protein (red) and non-supercharged natural protein corresponding thereto (black) to pDART plasmid, respectively, and expressing them, and detecting by western blotting, according to Example 15.

FIG. 19 shows the experimental result of measuring the degree of protein secretion in the enzyme activity measurement medium through the color change of the colony peripheral medium, after simultaneously expressing TliA protein (original substrate of TliDEF transporter) and T1SS transporters isolated from different 3 kinds of bacteria, according to Example 16.

FIG. 20 shows the experimental result of measuring the degree of protein secretion in the enzyme activity measurement medium through the color change of the colony peripheral medium, by suspending the LARD3 signal sequence to cutinase protein (Cuti) and highly negatively charged cutinase protein (Cuti(-)) and then expressing them together with different 3 kinds of T1SS transporter proteins in E. coli according to Example 17.

FIG. 21 shows the experimental result of detecting the protein concentration inside and outside the cell by western blotting, after attaching the LARD3 signal sequence to cutinase protein (Cuti) and highly negatively charged cutinase protein (Cuti(-)) and then expressing them together with different 3 kinds of T1SS transporter proteins in E. coli and liquid culturing them, according to Example 18.

FIG. 22 shows the experimental result of detecting the protein concentration inside and outside of the cell by western blotting, after attaching LARD3 signal sequence to M37 lipase protein (M37) and highly negatively charged M37 lipase protein (M37(-)), and then expressing them with different 3 kinds of T1SS transporter proteins in E. coli by performing liquid culture according to Example 19.

FIG. 23 shows the sequence identity between TliDET transporter and various T1SS transporters and the proportion of the portion of similar sequence in the full sequence.

FIG. 24 shows the amino acid sequences of wild type M37 and mutants with modified amino acids by using the selective superneutralization of the positive charges and the random mutagenesis-screening method.

FIG. 25 shows the experimental result of detecting the protein concentration inside and outside the cell by western blotting, measuring the degree of protein secretion in the enzyme activity measurement medium through the color change of the colony peripheral medium, and the mixed-based codon strategy utilized to prepare M37(var) mutant according to Examples 21 and 22.

### DETAILED DESCRIPTION OF THE EMBODIMENTS

Hereinafter, the present invention will be described in detail by examples. However, the following examples are intended to illustrate the present invention only, but the present invention is not limited by the following examples.

### [Example 1] Bacterial Strains and Growth Media

Plasmid construction and gene cloning were performed in E. coli XL1-BLUE. Protein expression and secretion were observed in the P. fluorescens ΔtliA ΔprtA strain, which is a double-deletion derivative of P. fluorescens SIK-W 1 (Son, M., Moon, Y., Oh, M. J., Han, S. B., Park, K. H., Kim, J G., and Ahn, J. H. (2012) Lipase and protease double-deletion mutant of Pseudomonas fluorescens suitable for extracellular protein production. Appl Environ Microbiol 78, 8454-8462). Microorganisms were cultured in lysogeny broth (LB) with 30 μg/ml kanamycin. An enzyme plate assay for the target genes with lipase activity (TliA, NKC-TliA, and CTP-TliA) was prepared with LB agar media containing blender-mixed 0.5% colloidal glyceryl tributyrate. E. coli and P. fluorescens were incubated at 37° C. and 25° C., respectively. E. coli transformation was performed following the standard heat-shock method, and P. fluorescens transformation was performed via electroporation at 2.5 kV, 125Ω, and 50 μF, with electrocompetent cells prepared using a standard electroporation protocol (Ausubel, M. F. (2014) Escherichia coli, Plasmids, and Bacteriphages. in Current Protocols in Molecular Biology, John Wiley & Sons, Inc. pp). The transformed P. fluorescens were cultured in test tubes with 5 ml of liquid LB media, including 60 μg/ml kanamycin, and were incubated at 25° C. in a 180 rpm shaking incubator until the stationary phase was reached. The proteins were analyzed for both expression and secretion by seeding the transformed cells in liquid LB or streaking them on the solid-plate activity assay.

### [Example 2] Plasmid Vector Constructions

Plasmid pDART was used for the secretory production of different proteins of the present inventors (Ryu, J., Lee, U., Park, J., Yoo, D. H., and Ahn, J. H. (2015) A vector system for ABC transporter-mediated secretion and purification of recombinant proteins in Pseudomonas species. Appl Environ Microbiol 81, 1744-1753). Plasmid vectors pFD10 and pBD10 were derivatives of pDART, constructed by adding codons for 10 aspartic acid residues to the target proteins in either the upstream or downstream position of MCS. The DNA sequence for 10 aspartic acids was amplified via PCR using synthesized Glycine max lunasin gene (Galvez, A. F., Chen, N., Macasieb, J., and de Lumen, B. O. (2001) Chemopreventive Property of a Soybean Peptide (Lunasin) That Binds to Deacetylated Histones and Inhibits Acetylation. Cancer Research 61, 7473-7478) as a template. Two different PCR products were obtained, each for pFD10 and pBD10. One or two arbitrary bases are inserted upstream or downstream of the primers to keep the translation in-frame, causing a slight size and pI difference between the pFD10- and pBD10-inserted proteins.

Then, recombining the PCR product with pDART to construct pFD10 and pBD10 was accomplished with an In-Fusion cloning kit (Clontech In-Fusion HD cloning plus CE). To linearize pDART, it was digested with either XbaI (pFD10 construction) or SasI (pBD10). Then, the linearized pDART and the corresponding PCR products were digested with In-Fusion 3′-to-5′-exodeoxyribonuclease and re-ligated following the standard protocol of the In-Fusion kit. Ligation of these DNA fragments with complementary ˜15-base 5′-overhangs resulted in pFD10 and pBD10 plasmid, ready for target gene insertions. pDART, pFD10, and pBD10 sequences near their MCSs are provided in Table 2.

The amino acid sequences underlined in the following Table 2 represent LARD3 signal sequences, and the bold “IEGR” is a residue that connects the target protein and LARD3 signal sequence, and is a part that Factor Xa recognizes and cleaves.

The target protein may be further purified from Factor Xa and LARD3 by purification tag such as His-tag.

The description of each part of the sequence of the following Table 2 was disclosed in FIG. 19a to FIG. 19g. FIG. 19a to FIG. 19f represent the total sequence of target proteins in FASTA format, and FIG. 19g represents color codes for indicating enzyme sites and polypeptide characteristics.

TABLE 2

| Full sequences of the target proteins, in FASTA formats | | SEQ ID NO |
|---|---|---|
| TliA, wild type | MGVFDYKNLGTEASKTLFADATAITLYTYHNLDNGFAVGYQQHGLGLGLPATLVGALLG | 1 |
| (as a reference) | STDSQGVIPGIPWNPDSEKAALDAVHAAGWTPISASALGYGGKVDARGTFFGEKAGYTT | |
| | AQAEVLGKYDDAGKLLEIGIGFRGTSGPRESLITDSIGDLVSDLLAALGPKDYAKNYAG | |
| | EAFGGLLKTVADYAGAHGLSGKDVLVSGHSLGGLAVNSMADLSTSKWAGFYKDANYLAY | |
| | ASPTQSAGDKVLNIGYENDPVFRALDGSTFNLSSLGVHDKAHESTTDNIVSFNDHYAST | |
| | LWNVLPFSIANLSTWVSHLPSAYGDGMTRVLESGFYEQMTRDSTIIVANLSDPARANTW | |
| | VQDLNRNAEPHTGNTFIIGSDGNDLIQGGKGADFIEGGKGNDTIRDNSGHNTFLFSGHF | |
| | GQDRIIGYQPTDRLVFQGADGSTDLRDHAKAVGADTVLSFGADSVTLVGVGLGGLWSEG | |
| | VLIS | |
| TliA, expressed | MSRMGVFDYKNLGTEASKTLFADATAITLYTYHNLDNGFAVGYQQHGLGLGLPATLVGA | 2 |
| in pDART plasmid | LLGSTDSQGVIPGIPWNPDSEKAALDAVHAAGWTPISASALGYGGKVDARGTFFGEKAG | |
| (this is used for | YTTAQAEVLGKYDDAGKLLEIGIGFRGTSGPRESLITDSIGDLVSDLLAALGPKDYAKN | |
| computational | YAGEAFGGLLKTVADYAGAHGLSGKDVLVSGHSLGGLAVNSMADLSTSKWAGFYKDANY | |
| analysis) | LAYASPTQSAGDKVLNIGYENDPVFRALDGSTFNLSSLGVHDKAHESTTDNIVSFNDHY | |
| | ASTLWNVLPFSIANLSTWVSHLPSAYGDGMTRVLESGFYEQMTRDSTIIVANLSDPARA | |
| | NTWVQDLNRNAEPHTGNTFIIGSDGNDLIQGGKGADFIEGGKGNDTIRDNSGHNTFLFS | |
| | GHFGQDRIIGYQPTDRLVFQGADGSTDLRDHAKAVGADTVLSFGADSVTLVGVGLGGLW | |
| | SEGVLISELIEGRGSDGNDLIQGGKGADFIEGGKGNDTIRDNSGHNTFLFSGHFGQDRI | |
| | IGYQPTDRLVFQGADGSTDLRDHAKAVGADTVLSFGADSVTLVGVGLGGLWSEGVLIS | |
| NKC-TliA: NKC | MSRHMGTAPKAMKLLKKLLKLQKKGIGSMGVFDYKNLGTEASKTLFADATAITLYTYHN | 3 |
| is marked cyan | LDNGFAVGYQQHGLGLGLPATLVGALLGSTDSQGVIPGIPWNPDSEKAALDAVHAAGWT | |
| | PISASALGYGGKVDARGTFFGEKAGYTTAQAEVLGKYDDAGKLLEIGIGFRGTSGPRES | |
| | LITDSIGDLVSDLLAALGPKDYAKNYAGEAFGGLLKTVADYAGAHGLSGKDVLVSGHSL | |
| | GGLAVNSMADLSTSKWAGFYKDANYLAYASPTQSAGDKVLNIGYENDPVFRALDGSTFN | |
| | LSSLGVHDKAHESTTDNIVSFNDHYASTLWNVLPFSIANLSTWVSHLPSAYGDGMTRVL | |
| | ESGFYEQMTRDSTIIVANLSDPARANTWVQDLNRNAEPHTGNTFIIGSDGNDLIQGGKG | |
| | ADFIEGGKGNDTIRDNSGHNTFLFSGHFGQDRIIGYQPTDRLVFQGADGSTDLRDHAKA | |
| | VGADTVLSFGADSVTLVGVGLGGLWSEGVLISELIEGRGSDGNDLIQGGKGADFIEGGK | |
| | GNDTIRDNSGHNTFLFSGHFGQDRIIGYQPTDRLVFQGADGSTDLRDHAKAVGADTVLS | |
| | FGADSVTLVGVGLGGLWSEGVLIS | |
| CTP-TliA: CTP | MSRMRGSHHHHHHGMASMTGGQQMGRDLYDDDDKDRWGSMYGRRARRRRRRSMAGTGGM | 4 |
| is marked cyan | GVFDYKNLGTEASKTLFADATAITLYTYHNLDNGFAVGYQQHGLGLGLPATLVGALLGS | |
| | TDSQGVIPGIPWNPDSEKAALDAVHAAGWTPISASALGYGGKVDARGTFFGEKAGYTTA | |
| | QAEVLGKYDDAGKLLEIGIGFRGTSGPRESLITDSIGDLVSDLLAALGPKDYAKNYAGE | |
| | AFGGLLKTVADYAGAHGLSGKDVLVSGHSLGGLAVNSMADLSTSKWAGFYKDANYLAYA | |
| | SPTQSAGDKVLNIGYENDPVFRALDGSTFNLSSLGVHDKAHESTTDNIVSFNDHYASTL | |
| | WNVLPFSIANLSTWVSHLPSAYGDGMTRVLESGFYEQMTRDSTIIVANLSDPARANTWV | |
| | QDLNRNAEPHTGNTFIIGSDGNDLIQGGKGADFIEGGKGNDTIRDNSGHNTFLFSGHFG | |
| | QDRIIGYQPTDRLVFQGADGSTDLRDHAKAVGADTVLSFGADSVTLVGVGLGGLWSEGV | |
| | LISELIEGRGSDGNDLIQGGKGADFIEGGKGNDTIRDNSGHNTFLFSGHFGQDRIIGYQ | |
| | PTDRLVFQGADGSTDLRDHAKAVGADTVLSFGADSVTLVGVGLGGLWSEGVLIS | |
| Mannanase | MSRHHHHHHTVSPVNPNAQQTTKAVMNWLAHLPNRTENRVLSGAFGGYSHDTFSMAEAD | 5 |
| (Mann) | RIRSATGQSPAIYGCDYARGWLETANIEDSIDVSCNSDLMSYWKNDGIPQISLHLANPA | |
| | FQSGHFKTPITNDQYKKILDSSTAEGKRLNTMLSKIADGLQELENQGVPVLFRPLHEMN | |
| | GERFWWGLTSYNQKDNERISLYKQLYKKIYHYMTDTRGLDHLIWVYSPDANRDFKTDFY | |
| | PGASYVDIVGLDAYFQDAYSINGYDQLTALNKPFAFTEVGPQTANGSFDYSLFINAIKH | |
| | RYPKTIYFLAWNDEWSPAVNKGASALYHDSWTLNKGEIWNGDSLTPIVEELIEGRGSDG | |
| | NDLIQGGKGADFIEGGKGNDTIRDNSGHNTFLFSGHFGQDRIIGYQPTDRLVFQGADGS | |
| | TDLRDHAKAVGADTVLSFGADSVTLVGVGLGGLWSEGVLIS | |
| Mussel adhesion | MSSMRGSHHHHHHGSASAKPSYPPTYKAKPSYPPTYKAKPSYPPTYKGCSSEEYKGGYY | 6 |
| protein (MAP): | PGNSNHYHSGGSYHGSGYHGGYKGKYYGKAKKYYYKYKNSGKYKYLKKARKYHRKGYKK | |
| used SpeI-SacI | YYGGSSEFAKPSYPPTYKAKPSYPPTYKAKPSYPPTYKELIEGRGSDGNDLIQGGKGAD | |
| insertion | FIEGGKGNDTIRDNSGHNTFLFSGHFGQDRIIGYQPTDRLVFQGADGSTDLRDHAKAVG | |
| | ADTVLSFGADSVTLVGVGLGGLWSEGVLIS | |
| Maltose binding | MSRKIEEGKLVIWINGDKGYNGLAEVGKKFEKDTGIKVTVEHPDKLEEKFPQVAATGDG | 7 |
| protein (MBP) | PDIIFWAHDRFGGYAQSGLLAEITPDKAFQDKLYPFTWDAVRYNGKLIAYPIAVEALSL | |
| | IYNKDLLPNPPKTWEEIPALDKELKAKGKSALMFNLQEPYFTWPLIAADGGYAFKYENG | |
| | KYDIKDVGVDNAGAKAGLTFLVDLIKNKHMNADTDYSIAEAAFNKGETAMTINGPWAWS | |
| | NIDTSKVNYGVTVLPTFKGQPSKPFVGVLSAGINAASPNKELAKEFLENYLLTDEGLEA | |
| | VNKDKPLGAVALKSYEEELAKDPRIAATMENAQKGEIMPNIPQMSAFWYAVRTAVINAA | |
| | SGRQTVDEALKDAQTRITKELIEGRGSDGNDLIQGGKGADFIEGGKGNDTIRDNSGHNT | |
| | FLFSGHFGQDRIIGYQPTDRLVFQGADGSTDLRDHAKAVGADTVLSFGADSVTLVGVGL | |
| | GGLWSEGVLIS | |
| Thioredoxin | MSRMLHQQRNQHARLIPVELYMSDKIIHLTDDSFDTDVLKADGAILVDFWAEWCGPCKM | 8 |
| (Trx) | IAPILDEIADEYQGKLTVAKLNIDQNPGTAPKYGIRGIPTLLLFKNGEVAATKVGALSK | |
| | GQLKEFLDANLAELIEGRGSDGNDLIQGGKGADFIEGGKGNDTIRDNSGHNTFLFSGHF | |
| | GQDRIIGYQPTDRLVFQGADGSTDLRDHAKAVGADTVLSFGADSVTLVGVGLGGLWSEG | |
| | VLIS | |
| Cutinase (Cuti) | MSRHHHHHHAPTSNPAQELEARQLGRTTRDDLINGNSASCADVIFIYARGSTETGNLGT | 9 |
| | LGPSIASNLESAFGKDGVWIQGVGGAYRATLGDNALPRGTSSAAIREMLGLFQQANTKC | |
| | PDATLIAGGYSQGAALAAASIEDLDSAIRDKIAGTVLFGYTKNLQNRGRIPNYPADRTK | |
| | VFCNTGDLVCTGSLIVAAPHLAYGPDARGPAPEFLIEKVRAVRGSALEELIEGRGSDGN | |
| | DLIQGGKGADFIEGGKGNDTIRDNSGHNTFLFSGHFGQDRIIGYQPTDRLVFQGADGST | |
| | DLRDHAKAVGADTVLSFGADSVTLVGVGLGGLWSEGVLIS | |
| Chitinase (Chi) | MSRHHHHHHANSPKQSQKIVGYFPSWGVYGRNYQVADIDASKLTHLNYAFADICWNGKH | 10 |
| | GNPSTHPDNPNKQTWNCKESGVPLQNKEVPNGTLVLGEPWADVTKSYPGSGTTWEDCDK | |
| | YARCGNFGELKRLKAKYPHLKTIISVGGWTWSNRFSDMAADEKTRKVFAESTVAFLRAY | |
| | GFDGVDLDWEYPGVETIPGGSYRPEDKQNFTLLLQDVRNALNKAGAEDGKQYLLTIASG | |
| | ASRRYADHTELKKISQILDWINIMTYDFHGGWEATSNHNAALYKDPNDPAANTNFYVDG | |
| | AINVYTNEGVPVDKLVLGVPFYGRGWKSCGKENNGQYQPCKPGSDGKLASKGTWDDYST | |
| | GDTGVYDYGDLAANYVNKNGFVRYWNDTAKVPYLYNATTGTFISYDDNESMKYKTDSIK | |
| | TKGLSGAMFWELSGDCRTSPKYSCSGPKLLDTLVKELLGGPINQKDTEPPTNVKNIVVT | |
| | NKNSNSVQLNWTASTDNVGVTEYEITAGEEKWSTTTNSITIKNLKPNTEYKFSIIAKDA | |
| | AGNKSQPTALTVKTDEANMTPPDGNGTATFSVTSNWGSGYNFSIIIKNNGTNPIKNWKL | |
| | EFDYSGNLTQVWDSKISSKTNNHYVITNAGWNGEIPPGGSITIGGAGTGNPAELLNAVI | |
| | SENELIEGRGSDGNDLIQGGKGADFIEGGKGNDTIRDNSGHNTFLFSGHFGQDRIIGYQ | |
| | PTDRLVFQGADGSTDLRDHAKAVGADTVLSFGADSVTLVGVGLGGLWSEGVLIS | |
| M37 lipase | MSRHMSYTKEQLMLAFSYMSYYGITHTGSAKKNAELILKKMKEALKTWKPFQEDDWEVV | 11 |
| (M37) | WGPAVYTMPFTIFNDAMMYVIQKKGAEGEYVIAIRGTNPVSISDWLFNDFMVSAMKKWP | |
| | YASVEGRILKISESTSYGLKTLQKLKPKSHIPGENKTILQFLNEKIGPEGKAKICVTGH | |
| | SKGGALSSTLALWLKDIQGVKLSQNIDISTIPFAGPTAGNADFADYFDDCLGDQCTRIA | |
| | NSLDIVPYAWNTNSLKKLKSIYISEQASVKPLLYQRALIRAMIAETKGKKYKQIKAETP | |
| | PLEGNINPILIEYLVQAAYQHVVGYPELMGMMDDIPLTDIFEDAIAGLLLEHHHHHHGT | |
| | ASELIEGRGSDGNDLIQGGKGADFIEGGKGNDTIRDNSGHNTFLFSGHFGQDRIIGYQP | |
| | TDRLVFQGADGSTDLRDHAKAVGADTVLSFGADSVTLVGVGLGGLWSEGVLIS | |
| Capsid (Cap), | MSRMARKKSTPRRRKAVKRRRTVRRRQSPKARVRSTTTKAKRRISPSGSGSQHLTVRKQ | 12 |
| Chaetoceros | PFSNATSQPKILDGALTSSLSRRLQNVIGLTNGNGGLGTEIMHIFFAPTLGIPLIAMNS | |
| salsugineum | AEGVALRPSSSADPFFIGFPGQTIKFDYVSSGTTPPATGNLVTFSNECGFSKWRIVSQG | |
| nuclear inclusion | LRMELANSDEENDGWFEAVRFNWRNNPADICFTPIDGTLGGAKTTDFAVAPSPVGMYAL | |
| virus (CsNIV | KDMAMVEQPGYTTGLLKDLKNHEFMLHPQSTTHDPIILEQSYEGTIGTTAADDMYYSVT | |
| | SEVFELGNTVRGNTMKNSLVDNNMDWIYLRLHCRTNNGTTSNGSKLIVNAIQNLEVSFN | |
| | PSSDFAAFQTINKMHPQQKKVDDQLNNSAEASNKRQKTGGGELIEGRGSDGNDLIQGGK | |
| | GADFIEGGKGNDTIRDNSGHNTFLFSGHFGQDRIIGYQPTDRLVFQGADGSTDLRDHAK | |
| | AVGADTVLSFGADSVTLVGVGLGGLWSEGVLIS | |
| DnaJ (Hsp40) | MSRMAKQDYYEILGVSKTAEEREIRKAYKRLAMKYHPDRNQGDKEAEAKFKEIKEAYEV | 13 |
| | LTDSQKRAAYDQYGHAAFEQGGMGGGGFGGGADFSDIFGDVFGDIFGGGRGRQRAARGA | |
| | DLRYNMELTLEEAVRGVTKEIRIPTLEECDVCHGSGAKPGTQPQTCPTCHGSGQVQMRQ | |
| | GFFAVQQTCPHCQGRGTLIKDPCNKCHGHGRVERSKTLSVKIPAGVDTGDRIRLAGEGE | |
| | AGEHGAPAGDLYVQVQVKQHPIFEREGNNLYCEVPINFAMAALGGEIEVPTLDGRVKLK | |
| | VPGETQTGKLFRMRGKGVKSVRGGAQGDLLCRVVVETPVGLNERQKQLLQELQESFGGP | |
| | TGEHNSPRSKSFFDGVKKFFDDLTRGTASELIEGRGSDGNDLIQGGKGADFIEGGKGND | |
| | TIRDNSGHNTFLFSGHFGQDRIIGYQPTDRLVFQGADGSTDLRDHAKAVGADTVLSFGA | |
| | DSVTLVGVGLGGLWSEGVLIS | |
| Endo-1,4-β- | MSRHHHHHHYKATTTRYYDGQEGACGCGSSSGAFPWQLGIGNGVYTAAGSQALFDTAGA | 14 |
| glucanase V | SWCGAGCGKCYQLTSTGQAPCSSCGTGGAAGQSIIVMVTNLCPNNGNAQWCPVVGGTNQ | |
| (Eg15) | YGYSYHFDIMAQNEIFGDNVVVDFEPIACPGQAASDWGTCLCVGQQETDPTPVLGNDTG | |
| | STPPGSSPPATSSSPPSGGGQQTLYGQCGGAGWTGPTTCQAPGTCKVQNQWYSQCLPGT | |
| | ASELIEGRGSDGNDLIQGGKGADFIEGGKGNDTIRDNSGHNTFLFSGHFGQDRIIGYQP | |
| | TDRLVFQGADGSTDLRDHAKAVGADTVLSFGADSVTLVGVGLGGLWSEGVLIS | |
| Green | MSRMSKGEELFTGVVPILVELDGDVNGHKFSVSGEGEGDATYGKLTLKFICTTGKLPVP | 15 |
| fluroescent | WPTLVTTFSYGVQCFSRYPDHMKRHDFFKSAMPEGYVQERTISFKDDGNYKTRAEVKFE | |
| protein (GFP) | GDTLVNRIELKGIDFKEDGNILGHKLEYNYNSHNVYITADKQKNGIKANFKIRHNIEDG | |
| | SVQLADHYQQNTPIGDGPVLLPDNHYLSTQSALSKDPNEKRDHMVLLEFVTAAGITHGM | |
| | DELIEGRGSDGNDLIQGGKGADFIEGGKGNDTIRDNSGHNTFLFSGHFGQDRIIGYQPT | |
| | DRLVFQGADGSTDLRDHAKAVGADTVLSFGADSVTLVGVGLGGLWSEGVLIS | |
| −30 Negatively | MSRMGHHHHHHGGASKGEELFDGVVPILVELDGDVNGHEFSVRGEGEGDATEGELTLKF | 16 |
| supercharged | ICTTGELPVPWPTLVTTLTYGVQCFSDYPDHMDQHDFFKSAMPEGYVQERTISFKDDGT | |
| GFP (GFP-(-30)) | YKTRAEVKFEGDTLVNRIELKGIDFKEDGNILGHKLEYNFNSHDVYITADKQENGIKAE | |
| | FEIRHNVEDGSVQLADHYQQNTPIGDGPVLLPDDHYLSTESALSKDPNEDRDHMVLLEF | |
| | VTAAGIDHGMDELYKELIEGRGSDGNDLIQGGKGADFIEGGKGNDTIRDNSGHNTFLFS | |
| | GHFGQDRIIGYQPTDRLVFQGADGSTDLRDHAKAVGADTVLSFGADSVTLVGVGLGGLW | |
| | SEGVLIS | |
| +36 Positively | MSRMGHHHHHHGGASKGERLFRGKVPILVELKGDVNGHKFSVRGKGKGDATRGKLTLKF | 17 |
| supercharged | ICTTGKLPVPWPTLVTTLTYGVQCFSRYPKHMKRHDFFKSAMPKGYVQERTISFKKDGK | |
| GFP (GFP-(+36)) | YKTRAEVKFEGRTLVNRIKLKGRDFKEKGNILGHKLRYNFNSHKVYITADKRKNGIKAK | |
| | FKIRHNVKDGSVQLADHYQQNTPIGRGPVLLPRNHYLSTRSKLSKDPKEKRDHMVLLEF | |
| | VTAAGIKHGRDERYKELIEGRGSDGNDLIQGGKGADFIEGGKGNDTIRDNSGHNTFLFS | |
| | GHFGQDRIIGYQPTDRLVFQGADGSTDLRDHAKAVGADTVLSFGADSVTLVGVGLGGLW | |
| | SEGVLIS | |
| Epidermal | MNSDSECPLSHDGYCLHDGVCMYIEALDKYACNCVVGYIGERCQYRDLKWWELRSRIEG | 18 |
| growth factor | RGSDGNDLIQGGKGADFIEGGKGNDTIRDNSGHNTFLFSGHFGQDRIIGYQPTDRLVFQ | |
| (EGF) | GADGSTDLRDHAKAVGADTVLSFGADSVTLVGVGLGGLWSEGVLIS | |
| Alkaline | MSSMPVLENRAAQGDITAPGGARRLTGDQTAALRDSLSDKPAKNIILLIGDGMGDSEIT | 19 |
| phosphatase (AP) | AARNYAEGAGGFFKGIDALPLTGQYTHYALNKKTGKPDYVTDSAASATAWSTGVKTYNG | |
| | ALGVDIHEKDHPTILEMAKAAGLATGNVSTAELQDATPAALVAHVTSRKCYGPSATSEK | |
| | CPGNALEKGGKGSITEQLLNARADVTLGGGAKTFAETATAGEWQGKTLREQAQARGYQL | |
| | VSDAASLNSVTEANQQKPLLGLFADGNMPVRWLGPKATYHGNIDKPAVTCTPNPQRNDS | |
| | VPTLAQMTDKAIELLSKNEKGFFLQVEGASIDKQDHAANPCGQIGETVDLDEAVQRALE | |
| | FAKKEGNTLVIVTADHAHASQIVAPDTKAPGLTQALNTKDGAVMVMSYGNSEEDSQEHT | |
| | GSQLRIAAYGPHAANVVGLTDQTDLFYTMKAALGLKELIEGRGSDGNDLIQGGKGADFI | |
| | EGGKGNDTIRDNSGHNTFLFSGHFGQDRIIGYQPTDRLVFQGADGSTDLRDHAKAVGAD | |
| | TVLSFGADSVTLVGVGLGGLWSEGVLIS | |
| Phospholipase | MSMSLSFTSAIAPAAIQPPMVRTQPEPLSSSQPVEASATKAPVATLSQNSLNAQSLLNT | 20 |
| A1 (PLA1) | LVSEISAAAPAAANQGVTRGQQPQKGDYTLALLAKDVYSTGSQGVEGFNRLSADALLGA | |
| | GIDPASLQDAASGFQAGIYTDNQQYVLAFAGTNDMRDWLSNVRQATGYDDVQYNQAVSL | |
| | AKSAKAAFGDALVIAGHSLGGGLAATAALATGTVAVTFNAAGVSDYTLNRMGIDPAAAK | |
| | QDAQAGGIRRYSEQYDMLTGTQESTSLIPDAIGHKITLANNDTLSGIDDWRPSKHLDRS | |
| | LTAHGIDKVISSMAEQKPWEAMANAHHHHHHGTASELIEGRGSDGNDLIQGGKGADFIE | |
| | GGKGNDTIRDNSGHNTFLFSGHFGQDRIIGYQPTDRLVFQGADGSTDLRDHAKAVGADT | |
| | VLSFGADSVTLVGVGLGGLWSEGVLIS | |

TABLE 3

| | | |
|---|---|---|
| Factor Xa | IEGR | 21 |
| LARD3 signal | GSDGNDLIQGGKGADFIEGGKGNDTIRDNSGH | 22 |
| peptide | NTFLFSGHFGQDRIIGYQPTDRLVFQGADGST | |
| | DLRDHAKAVGADTVLSFGADSVTLVGVGLGGL | |
| | WSEGVLIS | |
| pDART | MSRHMGTASELIEGRGSDGNDLIQGGKGADFI | 23 |
| Translation | EGGKGNDTIRDNSGHNTFLFSGHFGQDRIIGY | |
| Structure | QPTDRLVFQGADGSTDLRDHAKAVGADTVLSF | |
| | GADSVTLVGVGLGGLWSEGVLIS | |
| pFD10 | MSSDDDDDDDDDDSRHMGTASELIEGRGSDGN | 24 |
| Translation | DLIQGGKGADFIEGGKGNDTIRDNSGHNTFLF | |
| Structure | SGHFGQDRIIGYQPTDRLVFQGADGSTDLRDH | |
| | AKAVGADTVLSFGADSVTLVGVGLGGLWSEGV | |
| | LIS | |
| pBD10 | MSRHMGTASELDDDDDDDDDDDIEGRGSDGND | 25 |
| Translation | LIQGGKGADFIEGGKGNDTIRDNSGHNTFLFS | |
| Structure | GHFGQDRIIGYQPTDRLVFQGADGSTDLRDHA | |
| | KAVGADTVLSFGADSVTLVGVGLGGLWSEGVL | |
| | IS | |
| pBE10 | MSRHMGTASELEEEEEEEEEEGIEGRGSDGND | 26 |
| Translation | LIQGGKGADFIEGGKGNDTIRDNSGHNTFLFS | |
| Structure | GHFGQDRIIGYQPTDRLVFQGADGSTDLRDHA | |
| | KAVGADTVLSFGADSVTLVGVGLGGLWSEGVL | |
| | IS | |
| pBH10 | MSRHMGTASELHHHHHHHHHHGIEGRGSDGND | 27 |
| Translation | LIQGGKGADFIEGGKGNDTIRDNSGHNTFLFS | |
| Structure | GHFGQDRIIGYQPTDRLVFQGADGSTDLRDHA | |
| | KAVGADTVLSFGADSVTLVGVGLGGLWSEGVL | |
| | IS | |
| pBR10 | MSRHMGTASELRRRRRRRRRRGIEGRGSDGND | 28 |
| Translation | LIQGGKGADFIEGGKGNDTIRDNSGHNTFLFS | |
| Structure | GHFGQDRIIGYQPTDRLVFQGADGSTDLRDHA | |
| | KAVGADTVLSFGADSVTLVGVGLGGLWSEGVL | |
| | IS | |

Color code for enzyme sites and polypeptide features
>Multiple cloning site (MCS): XbaI: tctaga, SR NdeI:
catatg, HM KpnI: ggtacc, GT NheI: gctagc, AS SacI:
gagctc, E:

### [Example 3] Construction of Plasmids with Inserted Target Genes

Thirteen target genes were selected for pDART insertion. The genes were amplified with PCR from extracted genomic DNA samples (TliA, MBP, Trx, and Hsp40), total cDNA (Eg1V), synthesized DNA products (NKC-TliA, CTP-TliA, MAP, lunasin, lunasin derivatives, GFP, and supercharged GFPs), or plasmids (other proteins), or the like.

Their N-terminal signal peptides were detected with the SignalP 4.1 web-based prediction algorithm (http://www.cbs.dtu.dk/services/SignalP/) (Petersen, T. N., Brunak, S., von Heijne, G., and Nielsen, H. (2011) SignalP 4.0: discriminating signal peptides from transmembrane regions. Nature methods 8, 785-786) and were excluded from cloning and expression processes. For synthetic genes, the codons were optimized for either E. coli expression (supercharged GFPs) or P. fluorescens expression (TliA derivatives).

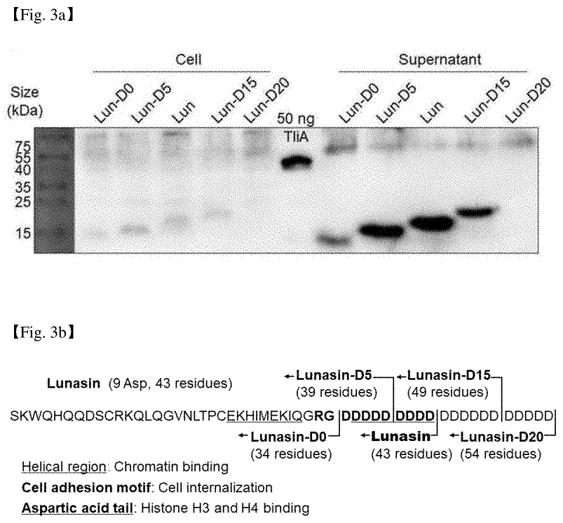

The lunasin gene was synthesized and amplified with PCR for pDART insertion. With various primers, we also synthesized its variations with differing lengths of Asp polypeptide tail at their C terminus such as lunasin-DO, lunasin-D5, lunasin-D15, and lunasin-D20 (FIG. 3B). NKC-TliA and CTP-TliA are derivatives of TliA. NKC is an antibiotic polypeptide developed previously, and CTP is a cytoplasmic transduction peptide that was developed as a cellular import tag previously. We have synthesized genes for these two, with codons optimized for P. fluorescens expression.

The supercharged variations of GFP, including negatively supercharged GFP (-30) and positively supercharged GFP (+36), were previously developed by replacing solvent-exposed residues of GFP with negatively or positively charged amino acids. We have completely synthesized genes that code for these two supercharged proteins, with codons optimized for E. coli expression.

The primers we used for PCR had restriction enzyme sites that were utilized to insert the target genes to the MCSs of the plasmids (pDART, pFD10, and pBD10). The PCR products and plasmid vectors were double-digested with two restriction enzymes for XbaI, KpnI, SacI, or SpeI (which is compatible with XbaI). The specific pair of enzymes used on each gene can be directly identified from the full sequences provided in Table 2.

Then, the plasmid treated by restriction enzyme and the gene were ligated with T4 ligase. The constructed plasmids were then introduced into E. coli for cloning, and the cloned plasmids were first obtained using a standard plasmid purification method. The purified plasmids were then introduced to P. fluorescens, for which expression and secretion were analyzed.

### [Example 4] Western Blotting Conditions

After 48 h of cell growth (secretion occurs during the entire growth), the liquid culture reached stationary growth phase, and the cell density reached about 1.5×10⁹ cells/ml (OD600=˜3). Then, 400 μl of the liquid cultures were taken and centrifuged at 18,000 rcf for 10 min to separate the supernatant and the cell pellet. 16 μl of culture (˜0.048 OD) equivalents of the cell pellet extract and supernatant were each loaded onto 10% polyacrylamide gels. SDS-PAGE was used to separate the proteins according to their sizes.

Then, the proteins were transferred to a nitrocellulose membrane (Amersham) for Western blotting. Polyclonal anti-LARDS rabbit immunoglobulin G (IgG) and anti-neomycin phosphotransferase 2 (Abcam, ab33595) were utilized as the primary antibody with 1:3000 and 1:500 dilution each, and anti-rabbit recombinant goat IgG-peroxidase (anti-rIgG goat IgG-peroxidase) was used as the secondary antibody with 1:1000 dilution. The bands were then detected using a chemiluminescence agent (Advansta WesternBright Pico). Western blotting images were acquired using an Azure C600 automatic detecting system. All included Western blotting images are representative results from at least three different repeated experiments, starting over again from cell culturing with independent P. fluorescens colonies.

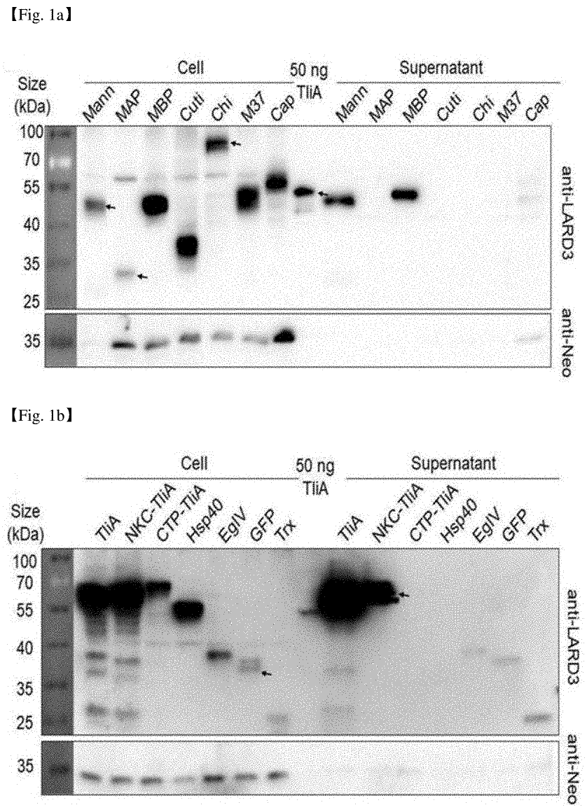

After the images were obtained, the results of experiment 1 (FIG. 1) was quantified with ImageJ software. Then, % secretion of the target proteins of this experiment was calculated. The % secretion was calculated as follows.

% secretion=Sₛᵤₚₑᵣₙₐₜₐₙₜ/(Sₛᵤₚₑᵣₙₐₜₐₙₜ+S_{cell})×100%

where S is the normalized signal strength of each bands in the Western blotting image, and the subscripts denote the sample type of the lanes.

### [Example 5] Analysis of Polypeptide Properties and Protein Structure

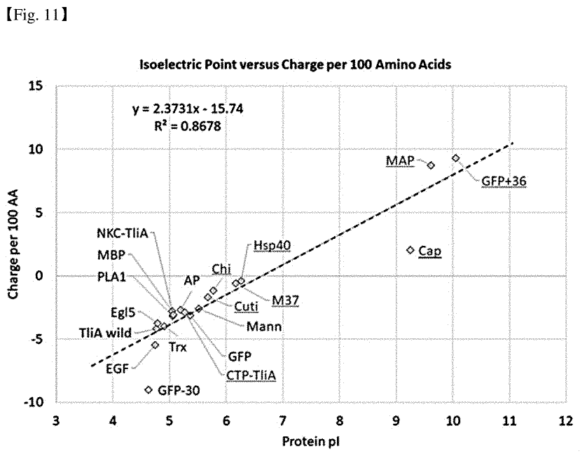

The theoretical pI values of the target proteins were calculated using the ExPASy Compute pI/Mw tool (Wilkins, M. R., Gasteiger, E., Bairoch, A., Sanchez, J. C., Williams, K. L., Appel, R. D., and Hochstrasser, D. F. (1999) Protein identification and analysis tools in the ExPASy server. Methods Mol Biol 112, 531-552). The entire sequences were used, and LARD3 and any additional sequences from the enzyme sites were included in the sequences for this purpose. The protein pI values are highly correlated with their charge per residue, and the correlation analysis of the protein pI values and their charge per residue is included in FIG. 11.

FIG. 11 shows relationship between protein pI and their charges at pH 7.0. Isoelectric points and charge per 100 residues of the LARD3-attached recombinant proteins show highly linear correlation. Wild-type TliA is marked in blue. Proteins that were observed not to be secreted to the extracellular culture were marked in red. As a result, a clear linear correlation is observed. The estimated unfolded protein charge at pH 7.0 is calculated by Protein Calculator v3.4 (http://protcalc.sourceforge.net/cgi-bin/protcalc).

Then, SWISS-MODEL structural homology modeling (https://swissmodel.expasy.org/) was used to study the ABC transporter protein structures (Arnold, K., Bordoli, L., Kopp, J., and Schwede, T. (2006) The SWISS-MODEL workspace: a web-based environment for protein structure homology modelling. Bioinformatics 22, 195-201).

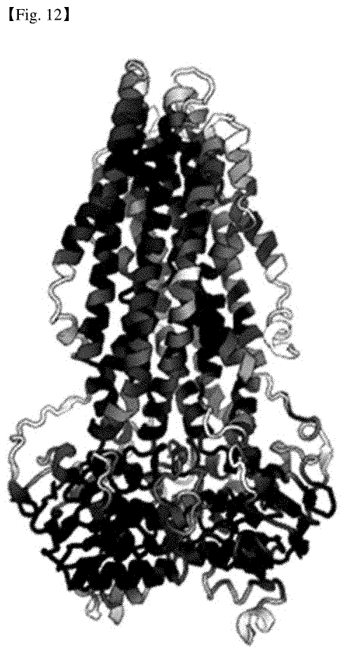

The present inventors used A. aeolicus PrtD (PDB code 5122) (Morgan, J. L. W., Acheson, J. F., and Zimmer, J. (2017) Structure of a Type-1 Secretion System ABC Transporter. Structure 25, 522-529) as a template, with sequence identity of 40.98%. The result of prediction of the structure of TliD was shown in FIG. 12.

FIG. 12 shows the structure prediction result of TliD and alignment with template, colored according to QMEAN4 score. Residues with low prediction degree (light color part of FIG. 12) are mainly located on the external surface, typically on random coils and protrusion parts. QMEAN4 score and coloring were obtained by SWISS-MODEL.

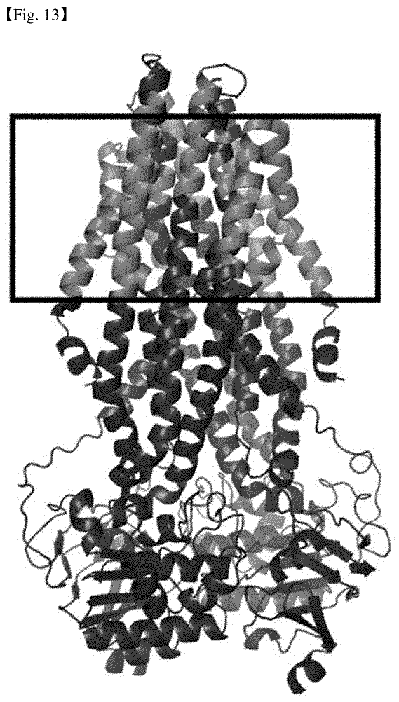

The model's transmembrane helices were verified by DAS-TMfilter (http://mendel.imp.ac.at/sat/DAS/) (Cserzo, M., Eisenhaber, F., Eisenhaber, B., and Simon, I. (2002) On filtering false positive transmembrane protein predictions. Protein Engineering, Design and Selection 15, 745-752), and the results are provided in FIG. 13.

FIG. 13 shows transmembrane helices prediction result of modeled TliD. The prediction was obtained by DAS-TMfilter webserver. (A) Predicted structure of TliD dimer, with transmembrane helices marked with different colors. (B) Sequence of TliD, highlighted with the identical color-codes with (A).

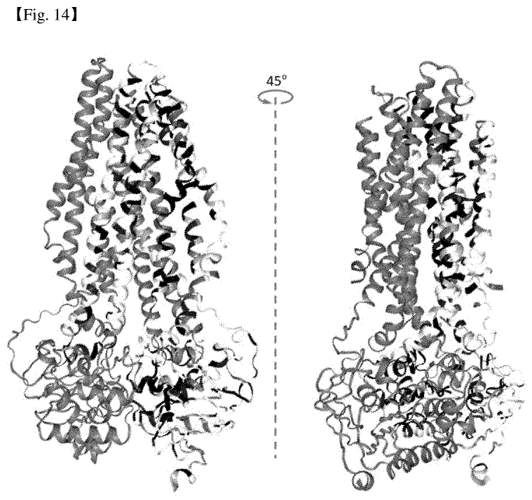

The surface of the obtained 3D model was calculated with Swiss PdbViewer (spdbv) (http://spdbv.vital-it.ch/) and colored according to the charge. The present inventors used the ConSurf web server (http://consurf.tau.ac.il/2016/) to compare TliD with its homologs and to verify the structure prediction of TliD (Ashkenazy, H., Abadi, S., Martz, E., Chay, O., Mayrose, I., Pupko, T., and Ben-Tal, N. (2016) ConSurf 2016: an improved methodology to estimate and visualize evolutionary conservation in macromolecules. Nucleic Acids Research 44, W344-W350). FIG. 14 includes information of conserved residues of TliD.

FIG. 14 shows the Consurf homologue conservation analysis result of modeled TliD. The present inventors ran a ConSurf homolog conservation analysis on TliD. Multiple Sequence Alignment were built using MAFFT, the homologues were collected from UniProt, homologue search algorithm BLAST, PSI-BLAST E-value 0.001, number of PSI-BLAST Iterations 5, % ID Between Sequences 25-95%. 197 unique proteins were scanned, and among them, 50 sequences closest to the query were used. Phylogenic neighbors were scanned with ML distance, and the conservation score was calculated with Bayesian algorithm. (A) TliD dimer, colored according to the Bayesian conservation score. The transmembrane helices of TliD were conserved among the homologues. Specifically, the residues facing inside of the TliD's central channel were highly conserved, while residues facing outside of the central channel (facing the phospholipids or the cytoplasm) were highly variable.

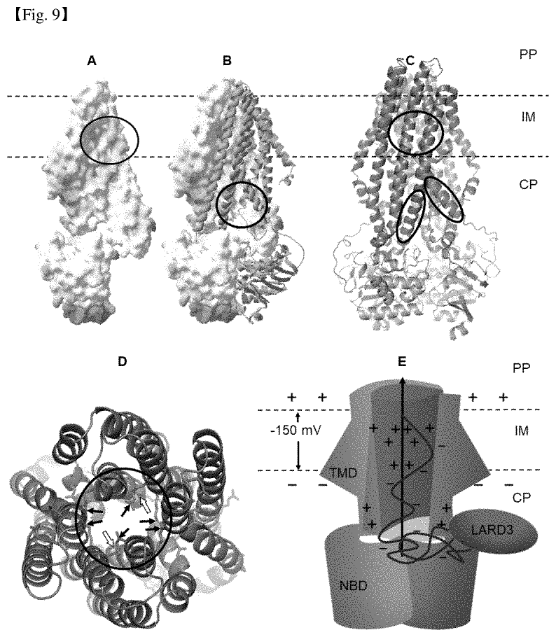

The ConSurf homology analysis also approved our structural prediction in a sense that most of the transmembrane helices were highly conserved in the inner surface-facing residues, and this makes our structural prediction even more persuasive. Finally, the present inventors checked side-chain pKa values of the highly conserved arginine and lysine residues at the potentially important positions (C, D and F of FIG. 9) with the web-based PDB 2PQR server (http://nbcr-222.ucsd.edu/pdb2pqr_2.0.0/).

In the subsequently progressed homology-based structure prediction model, it was shown that the dimer of this protein has positive charge distribution at the inner surface of the channel (FIG. 9A and FIG. 9B). This prediction model was prepared using Aquifex aeolicus PrtD (PDB code 5122) with sequence identity of 40.98% as a template. Moreover, a ConSurf homolog conservation analysis on TliD showed that these charges were indeed conserved, forming a positively charged sub-region at the midpoint of the channel (FIG. 9C and FIG. 9D). In addition, on the kinked helix on the substrate entry window, there is a positively-charged residue that sticks out toward the pore of the window and blocks the window in ADP-bound state of TliD. The ConSurf results also verified that this residue was charge-conserved, as all of the 50 homologs had either arginine or lysine at this residue (FIG. 9C, black arrow). The present inventors expect that this positively-charged inner surface interacts with negatively-charged residues during protein transport, facilitating secretion (FIG. 9E).

FIG. 9 shows the charge distribution in the structure of TliD, the ABC protein of the TliDEF complex. (A) electron repulsion surface of the TliD monomer. Colored according to its surface electric potential, from blue (+7 kBT/e) to red (-7 kBT/e). The inner surface of the central channel with highly positive charge is circled on top. (B) TliD homodimer, with one of the monomers presented in the ribbon model. The inner surface of the central channel with positive charge is circled on top and substrate entry window is circled on bottom. (C) TliD. The conserved positive charge cluster at the midpoint of the channel's inner surface are circled. The two α-helices that form substrate entry window are ovaled. Among the two conserved positively-charged residues, Arg-316 (black arrow) sticks out to the pore. (D) TliD dimer, seen from the periplasmic face. Positive charges are located in the middle of the channel (circled yellow), whereas negative charges are outside of the channel (E) schematic model of the TliD dimer, transporting a highly negatively charged recombinant polypeptide with the attached LARD3. The NBD (nucleotide-binding domain) and transmembrane domain (TMD) of TliD are labeled accordingly. The electric potential across the inner membrane (IM) is −150 mV, where the cytoplasm (CP) is more negative than the periplasm (PP). This potential difference also makes it more favorable to outward-transport negatively-charged proteins than positively-charged proteins.

The present inventors visualized the results with the PyMOL software. All sequences that were used for the analysis are provided in Table 2 and Table 3.

### [Example 6] Cross-Analyzing the Secretion of Recombinant Proteins and their pI

Thirteen genes (Table 4) of target proteins of different sizes, flexibility, volume, weight, etc. were introduced to P. fluorescens ΔtliA ΔprtA via pDART, where they are attached to a C-terminal LARD3 signal sequence. Table 4 represents the list of genes and their references, and genes indicated by * represent genes which were not used in the present experiment, but were secreted in the previous research. Then, after liquid culturing the cells, the supernatant and cell pellet were analyzed via Western blotting (FIG. 1a and FIG. 1b).

TABLE 4

| Code | Protein name | Source | DNA type |
|---|---|---|---|
| TliA | Thermostable | Pseudomonas fluorescens SIK-W1 | Genomic |
| | lipase A | | DNA |
| NKC- | NKC-TliA | Yang, K. S., Sung, B. H., Park, M. K., | Synthesized |
| TliA | | Lee, J. H., Lim, K. J., Park, S. C., | |
| | | Kim, S. J., Kim, H. K., Sohn, J. -H., | |
| | | Kim, H. M., and Kim, S. C. (2015) | |
| | | Recombinant Lipase Engineered with | |
| | | Amphipathic and Coiled-Coil | |
| | | Peptides. ACS Catalysis 5, 5016-5025 | |
| CTP- | CTP-TliA | Kim, D., Jeon, C., Kim, J. -H., Kim, | Synthesized |
| TliA | | M. -S., Yoon, C. -H., Choi, I. -S., Kim, | |
| | | S. -H., and Bae, Y. -S. (2006) | |
| | | Cytoplasmic transduction peptide | |
| | | (CTP): New approach for the delivery | |
| | | of biomolecules into cytoplasm in | |
| | | vitro and in vivo. Experimental Cell | |
| | | Research 312, 1277-1288 | |
| Mann | Mannanase | Bacillus subtilis | Plasmid |
| MAP | Mussel adhesion | MAP fp-151 | Synthesized |
| | protein | | |
| MBP | Maltose binding | Escherichia coli XL1-Blue | Genomic |
| | protein | | DNA |
| Trx | Thioredoxin | Escherichia coli XL1-Blue | Genomic |
| | | | DNA |
| Cuti | Cutinase | Nectria haematococca | Plasmid |
| Chi | Chitinase | Bacillus thuringenesis | Plasmid |
| M37 | M37 lipase | Photobacterium lipolyticum | Plasmid |
| Cap | Capsid protein | Chaetoceros salsugineum | Plasmid |
| | | DNA inclusion virus | |
| Hsp40 | DnaJ charperone | Escherichia coli XL1-Blue | Genomic |
| | | | DNA |
| EglV | Endo-1,4-β- | Trichoderma reesei QM6a | Total |
| | glucanase V | | cDNA |
| GFP | Green fluorescent | pGFPuv (Clontech) | Plasmid |
| | protein | | |
| GFP(−30) | Negatively | Lawrence, M. S., Phillips, K. J., and | Synthesized |
| | supercharged GFP | Liu, D. R. (2007) Supercharging | |
| | | proteins can impart unusual resilience. | |
| | | Journal of the American Chemical | |
| | | Society 129, 10110-10112 | |
| GFP(+36) | Positively | Lawrence, M. S., Phillips, K. J., and | Synthesized |
| | supercharged GFP | Liu, D. R. (2007) Supercharging | |
| | | proteins can impart unusual resilience. | |
| | | Journal of the American Chemical | |
| | | Society 129, 10110-10112 | |
| EGF | Epidermal growth | Homo sapiens | Plasmid |
| | factor | | |
| AP | Alkaline phosphatase | Escherichia coli XL1-Blue | Genomic |
| | | | DNA |
| PLA1 | Phospholipase A₁ | Serracia marescens | Plasmid |

As shown in FIG. 1a and FIG. 1b, Mannanase, MBP, NKC-TliA, Eg1V, GFP, and thioredoxin were both detectable in the cell pellet and the supernatant, showing successful expression and secretion out to the extracellular media. However, MAP, cutinase, chitinase, capsid, Hsp40, and CTP-TliA were not detected in the supernatant despite being detected in the cell pellet, signifying that they were not secreted.

FIG. 1a and FIG. 1b confirm secretion of selected proteins, and represent western blotting image showing the expression and secretion of the target proteins. The cell samples show the amount of the protein that remains in the cytoplasm, and the supernatant samples represent the amount of protein that is localized to the extracellular space. For comparison, equivalent amounts of cell extract and culture supernatant (16 μl) were loaded onto the gel and were analyzed via Western blotting. 50 ng of TliA was loaded in the middle of the gel as a reference. Two other Western blottings were obtained from different culture samples. All of the unpresented results exhibit similar patterns. Below the images, there are Western blottings of the same samples but with primary antibody against cytosolic Neo, the neomycin/kanamycin phosphotransferase 2 protein. The nonspecific lysis or leakage is minimal in all samples except capsid.

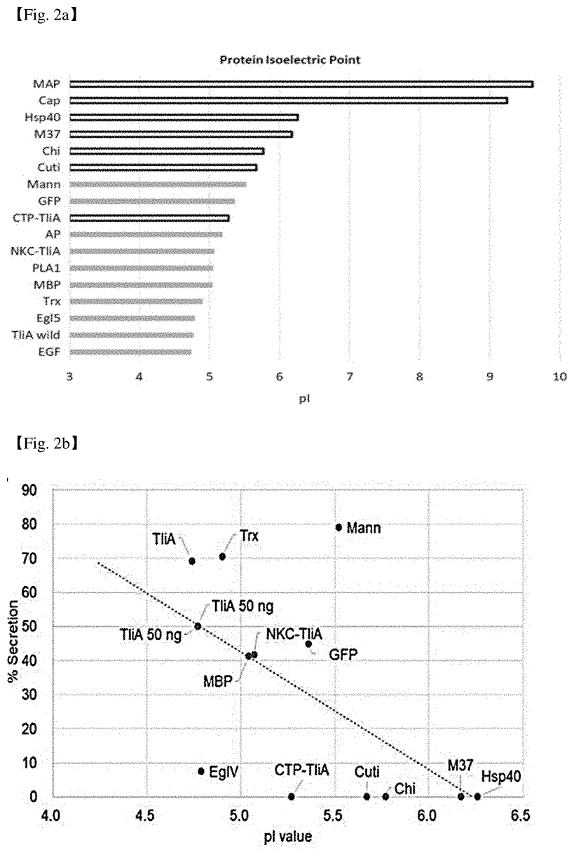

These non-secreted proteins have a relatively high theoretical pI. All of them (with one exception, CTP-TliA) were above ˜5.5, being either positively or less negatively charged. In contrast, the secreted proteins were relatively acidic and highly negatively charged with a pI that does not exceed 5.5 (FIG. 2a and FIG. 2b).

FIG. 2a and FIG. 2b shows correlation between % secretion of the target proteins and their pI values. The pI value of the target proteins is calculated from the sequence. The proteins that have not been secreted have their bars colored red. AP, EGF, and PLA1 are proven to be secreted in previous studies and are added in this figure. FIG. 2b added secretion percentage to pI. Three different biological replicates (independent culture samples) of the experiment in FIG. 1 were used for the quantitative analysis. Two highly basic outlier proteins that were not secreted, MAP (pI=9.61) and capsid (pI=9.25), were excluded from the plot. There was a negative correlation between the protein pI and their % secretion.

As could be seen in FIG. 1b and FIG. 2b, the secretion of NKC-TliA and CTP-TliA decreased dramatically from that of original TliA. These are derivatives of TliA with an N-terminally attached short, extremely positively-charged sequence (Table 2). CTP-TliA was not secreted at all. Note that CTP has nine consecutive residues composed solely of arginine with only one exception, alanine (RRARRRRRR), as described in Table 2.

Then, after quantification of western signal strengths of proteins, the percentage of secreted protein versus the total amount of expressed protein was plotted. The result was shown in FIG. 2b.

As shown in FIG. 2b, there seemed to be a weak negative correlation between protein pI and their secretion efficiency, but there were also a few exceptions.

### [Example 7] Analysis of Lunasin and its Derivatives

Lunasin is an anticancer polypeptide from soybean Glycine max. It has a unique feature of nine consecutive aspartate (aspartic acid, Asp) sequences at its C terminus. The present inventors have constructed multiple derivatives of lunasin with different lengths of the aspartate polypeptide tails.

Then, lunasin and its derivatives were introduced to P. fluorescens via pDART, and their expression and secretion were observed via Western blotting, and the result was shown in FIG. 3a and FIG. 3b.

FIG. 3a and FIG. 3b is the result of confirming expression and secretion of lunasin and its derivatives with different lengths of the oligo-aspartic acid tails, to determine the optimal length of the oligo-aspartic acid sequence in P. fluorescens expression and secretion system.

FIG. 3a detected expression of lunasin and its derivatives in the cell and supernatant via Western blotting, and specifically, 36-μl eq of cell extract and supernatant were loaded onto the gel and were analyzed via Western blotting. FIG. 3b represents protein sequence and domain structure of lunasin and its derivatives whose length of the aspartic acid tail is modified, and they were named as lunasin-D0, lunasin-D5, original lunasin (D9), lunasin-D15, and lunasin-D20, respectively.

As shown in FIG. 3a, the original lunasin showed that the highest secretion and relative amount of secreted proteins declined as the length of the oligo-aspartate tail decreased. The present inventors have also observed decreased secretion and expression levels in lunnasin-D15. Lunasin-D20 was not expressed in either the cell or supernatant. The exact sequence of the lunasin polypeptide and its derivatives is given in FIG. 3b.

Based on this experiment, the present inventors determined that the optimal length of the aspartate polypeptide sequence would be approximately nine, and we set up the experiments below.

### [Example 8] Construction of pFD10 and pBD10 with Added Aspartate Polypeptide

Among the 20 most common amino acids, aspartic acid has the lowest side chain pKa value (Mathews, C. K. (2013) Biochemistry, 4th ed., Pearson, Toronto). Inspired by the lunasin protein sequence in the Example 7, the present inventors developed two plasmids that add the aspartate polypeptide sequence to the inserted proteins as well as the LARD3 signal sequence.

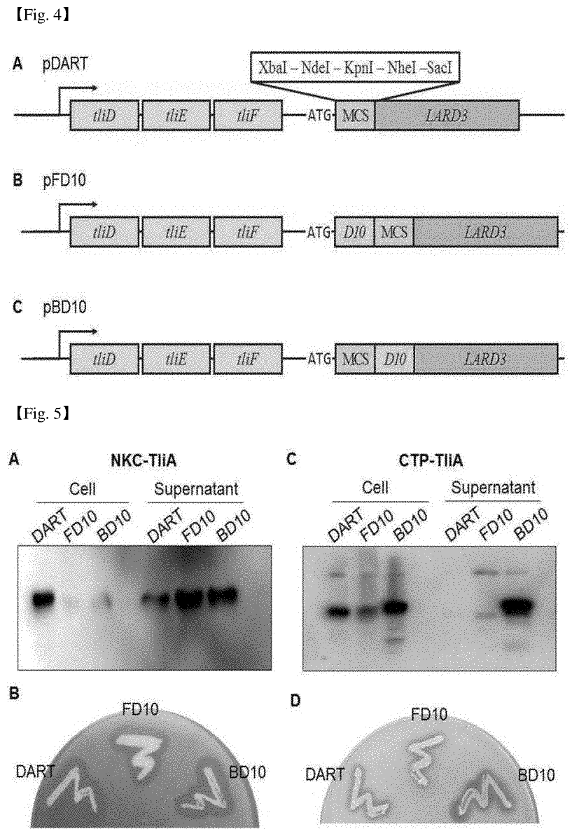

The present inventors have synthesized an aspartate-decamer-coding DNA sequence based on the DNA sequence of the lunasin gene's aspartate polypeptide tail, and have named D10 (DDDDDDDDDD: SEQ ID NO: 33). Then, the present inventors conjugated D10 to the pDART plasmid, creating two types of plasmid that either add D10 to the N terminus or to the C-terminus of the gene inserted to MCS, respectively, by inserting into pDART plasmid (named pGD10 and pBD10, respectively), and this was shown in FIG. 4.

FIG. 4 shows the structures of plasmids used, and represents the structure of pDART plasmid comprising MCS. tliD, tliE, tliF, and the LARD3-attached fusion protein are controlled in a single operon. (A) MCS is directly followed by the LARD3 gene, and thus the inserted target gene is expressed with LARD3 attached on its C terminus. (B) represents structure of pFD10 plasmid that attaches D10 sequence at the N terminus. The D10 gene directly follows the start codon and is located right before the MCS and LARD3. (C) represents structure of the pBD10 plasmid, which attaches D10 sequence at the C-terminal side, but before LARD3. The D10 gene is located between the MCS and LARD3.

Then, selected proteins were inserted into both of the newly created plasmids, pFD10 and pBD10. These pFD10 or pBD10-cloned recombinant proteins were introduced to P. fluorescens alongside their pDART-cloned counterparts, and the secretion efficiency was analyzed via Western blot analysis.

### [Example 9] Insertion of TliA-Derived Recombinant Proteins into pFD10 and pBD10

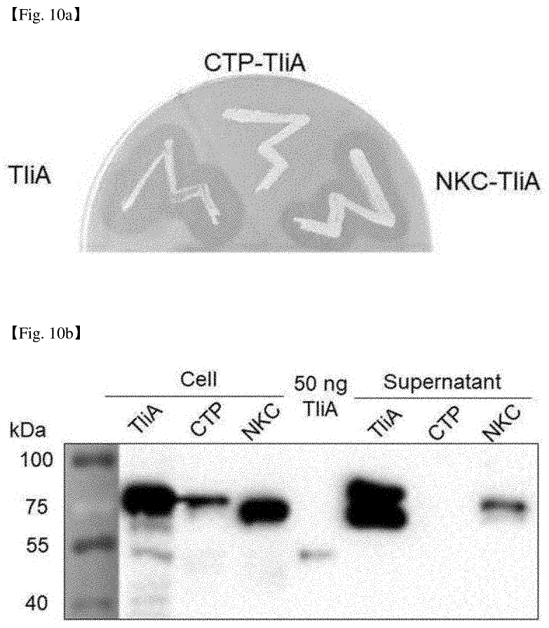

NKC-TliA and CTP-TliA are both derivatives of TliA, each with an N-terminal basic-peptide attachment. Their secretion efficiency through TliDEF is significantly smaller than wild-type TliA (FIG. 1b and FIG. 10a, b).

FIG. 10a shows the result of enzyme plate assay of TliA, CTP-TliA and NKC-TliA, and TliA was secreted as expected (TliA is the natural substrate for the TliDEF transporter). However, secretion of CTP-TliA is blocked, and secretion of NKC-TliA is somewhat weaker than TliA.

FIG. 10b represents the result of western blotting of TliA, CTP-TliA, and NKC-TliA, and as could be seen in the enzyme plate assay, the secretion of TliA is strong, NKC-TliA is only weakly secreted, and CTP-TliA is not secreted. NKC is highly positively charged, and CTP has even more positively charged. CTP carries a consecutive nine residues composed solely of arginine with one exception in the middle, alanine.

However, the oligo-aspartate attachment on them by pFD10 or pBD10 greatly re-increases their secretion. The experimental result was shown in FIG. 5.

FIG. 5 shows the result of adding 10 aspartic acids to the N-terminus (1-D10) and C-terminus (BD10) of two kinds of TliA lipases in which NKC and CTP sequences are attached, respectively (NKC-TliA, CTP-TliA) and expressing through pDART plasmid, then detecting by western blotting and lipase activity measuring medium.

In (A) of FIG. 5, secretion strongly improved in both pFD10 and pBD10 when compared with pDART. (B) represents the result of enzyme plate assay of NKC-TliA in different plasmids. (C) is the result of western blotting of CTP-TliA in pFD10 and pBD10, and it is confirmed that secretion strongly increased in pBD10. (D) represents the result of enzyme plate assay of CTP-TliA in different plasmids. pBD10 exhibits a major increase in secretion. Two other Western blot results were obtained from different culture samples, and both of them exhibit similar patterns. Two other enzyme plate assays were obtained from different colonies, and both of them exhibit similar patterns.

In terms of the secretion ratio (secreted protein versus intracellular protein), NKC-TliA shows a dramatic increase in secretion after the addition of either an upstream or downstream D10 sequence, as shown in (A) and (B) of FIG. 5.

CTP-TliA also shows a drastic increase in secretion in both the Western blotting and activity plate assays when a downstream D10 sequence was added by pBD10, as shown in (C) and (D) of FIG. 5. In enzyme plate activity assays, the halo sizes of NKC-TliA and CTP-TliA in pDART or pBD10 are generally consistent with the band strength of the supernatant samples in their respective Western blotting results. However, pFD10 has a slightly smaller halo than expected from their band strength, indicating the possibility of a reduced enzymatic activity.

### [Example 10] Insertion of Negatively-Charged Proteins to pFD10 and pBD10

A recombinant plasmid obtained by introducing genes for GFP, mannanase, maltose binding protein (MBP), and thioredoxin to pDART, pFD10, and pBD10 was introduced to P. fluorescens to prepare transformed proteins. The produced proteins were secreted by the TliDEF transporter, and the experimental result was shown in FIG. 6.

FIG. 6 shows secretion of negatively-charged proteins in pFD10 and pBD10. (A) represents the result of western blotting of GFP, and both pFD10 and pBD10 exhibit an increase in protein secretion in the supernatant. (B) represents the result of western blotting of Mannanase, and both pFD10 and pBD10 exhibit slight increases in mannase secretion. (C) represents the result of western blotting of MBP, and the increased secretion ratio was observed in both pFD10 and pBD10. (D) represents the result of western blotting of thioredoxin, and the signals were weak overall, but there was an increase in the secretion for both pFD10 and pBD10. Overall, the bands of more negatively-charged proteins in pBD10 appeared in slightly upward-shifted positions. Three other Western blot results for pDART and pBD10 were from different culture samples were obtained, whereas there were two other Western blot results for pDART and pFD10. All of them exhibit similar patterns.

As shown in FIG. 6, GFP showed the most dramatic increase in the increase of secretion. A comparison of the band strength of pDART and pBD10-inserted GFP showed a remarkable change in the supernatant versus cell expression ratio. pFD10-GFP also exhibited some improvement in terms of the ratio between the supernatant and the cell pellet ((A) of FIG. 6).

The case of mannanase was somewhat vague, but it could be concluded that pBD10-mannanase exhibits a better secretion than pDART-mannanase. In addition, although the absolute expression itself decreased, there was a small improvement in the ratio when an upstream D10 sequence was added by pFD10 ((B) of FIG. 6).

The secretion of MBP improved in both pFD10 and pBD10 in terms of the supernatant/cell ratio, compared with pDART ((C) of FIG. 6).

In the case of Trx (thioredoxin), the supernatant/cell ratio improved in pFD10 and pBD10 ((D) of FIG. 6).

Consequently, as the result of western blotting, it was confirmed that proteins in which aspartic acid was added to the N-terminus or C-terminus had increased (Supernatant) to intracellular (Cell) protein concentration ratio, and FD10 showed a significantly reduced pattern of expression.

### [Example 11] Addition of Positively Charged Amino Acid Oligomers—Construction and Analysis of pBR10

The present inventors constructed an additional plasmid that closely resembles pBD10, but with one difference. In this plasmid, the D10 sequence, the DNA sequence that codes for aspartate oligomer, was replaced with R10 that codes for arginine oligomer.

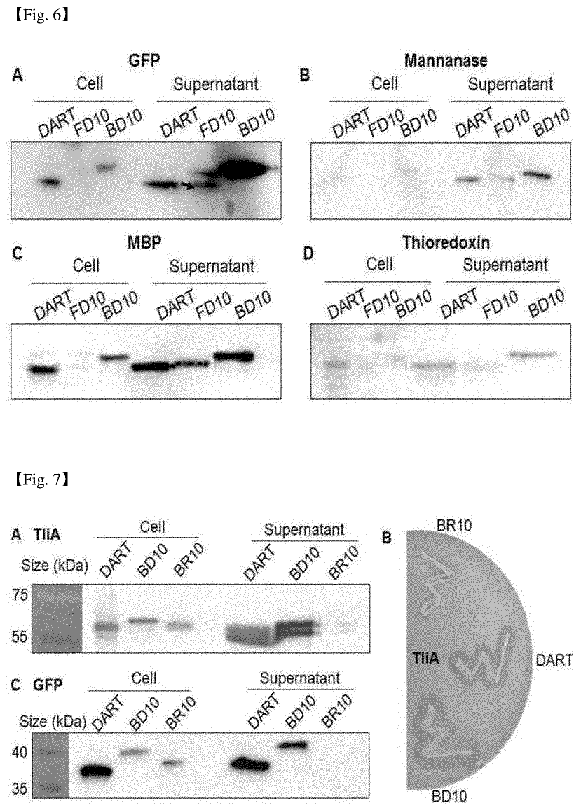

The present inventors inserted the TliA and GFP gene to pDART, pBD10, and pBR10 plasmids and examined their secretion by enzyme activity media (TliA only) and Western blotting, and the results were shown in FIG. 7.

FIG. 7 represents the result of adding 10 aspartic acids (D) or 10 arginines, respectively, at the C-terminus of TliA lipase and green fluorescent protein (GFP) and then detecting by western blotting and lipase activity media. negatively-charged proteins, TliA and GFP, were inserted in the plasmids that attach nothing except the signal sequence (pDART), oligo-aspartate (pBD10), and oligo-arginine (pBR10). A of FIG. 7 represents the result of western blot of TliA in these plasmids. TliA in pDART and pBD10 shows good secretion. However, the secretion was blocked when R10 was attached. B of FIG. 7 represents the result of enzyme plate assay of TliA in these plasmids. Secretion of TliA was blocked when it was inserted to pBR10.

In Western blotting of TliA, pDART and pBD10 exhibited good secretion efficiency. pBR10, however, blocked the secretion (A of FIG. 7). Similar patterns were observed in enzyme plate assay, pBR10 did not exhibit halo, but the others did (B of FIG. 7). In Western blotting of GFP, both pDART and pBD10 exhibited secretion. Yet again, pBR10 blocked secretion of GFP as it did to TliA (C of FIG. 7).

### [Example 12] Western Blot Analysis of Supercharged Proteins

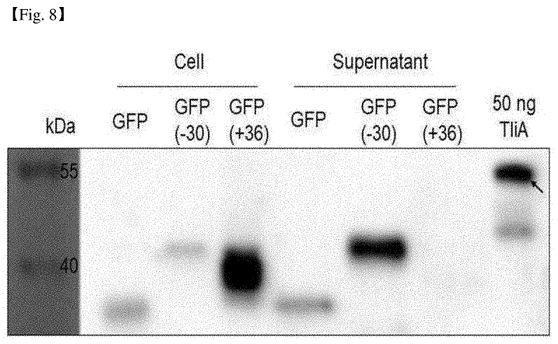

Green fluorescent protein (GFP) and its two supercharged derivatives, GFP (-30) and GFP (+36) by Average Number of Neighboring Atoms Per Sidechain Atom (AvNAPSA) (Lawrence M S, Phillips K J, Liu D R. Supercharging Proteins Can Impart Unusual Resilience. Journal of the American Chemical Society 2007; 129: 10110-10112.) method, were recombined with LARDS through pDART and introduced to P. fluorescens ΔtliA ΔprtA, to express proteins, and then the samples were analyzed via western blotting, and the result was shown in FIG. 8.

FIG. 8 represents secretion of GFP and supercharged GFPs. GFP (-30) exhibited a much higher extracellular (Supernatant) to intracellular (Cell) protein concentration ratio, and a significantly higher secretion than the original GFP.

On the other hand, positively supercharged GFP (+36) was detected in cells at a high concentration, but was not secreted at all outside of the cell (Supernatant). Although the bands of the supercharged GFPs are also slightly shifted upwards, but two other Western blot results from different culture samples were obtained, and both of them exhibit similar patterns.

As could be seen in FIG. 8, both GFP and GFP (-30) were detected in the cell pellet and the supernatant, indicating that they were effectively expressed and secreted to the extracellular space. Herein, it could be confirmed that GFP (-30) was more strongly localized to the supernatant than the original GFP.

In contrast, GFP (+36) was heavily expressed but localized in the cell pellet and was not secreted to the extracellular space. The pI values for these recombinant proteins were 4.64 for GFP (-30), 5.36 for unmodified GFP, and 10.42 for GFP (+36).

### [Example 13] Confirmation of the Optimal Linker Length for Increasing the Protein Secretion Efficiency

NKC-TliA was selected as a model protein. NKC consists of 21 amino acids, and is a peptide forming amphiphilic α-helix. 21 amino acids include lysine a lot, and as pI=10.78, when NKC-TliA is prepared by fusion to TliA lipase in which pI=5.01, pI=5.34 and the protein secretion is reduced.

The present inventors have confirmed that the secretion by replacing all the lysine of NKC protein to aspartate (NKC(-)), and in addition, have compared the secretion efficiency of NKC(-) through various linker lengths by linking linkers with various lengths to NKC(-) and TliA through western blotting and activity analysis plate, and the results were shown in FIG. 15. The lengths of linker were represented by L1 with one GGGGS from NKC (-) where noting is present, L2 with 2, and L3 with 3.

FIG. 15 represents the protein secretion in TliA, NKC(-), NKC-L1, NKC-L2, NKC-L3, and NKC-TliA. (A) of FIG. 15 is the western blotting result of TliA, and (B) of FIG. 15 shows the result of enzyme plate analysis of TliA in other plasmids.

As the result of the western blotting of (A) of FIG. 15, it could be confirmed that the far right NKC-TliA was not secreted at all, but secretion of NKC(-) in which all the lysine was replaced with aspartate and the protein in which a linker was attached to negatively charged NKC was significantly increased.

According to the result of the activity analysis plate of (B) of FIG. 15, it could be confirmed that the protein secretion was increased in NKC(-) than the conventional NKC, and the secretion was increased overall when a linker was introduced, and in particular, when 3 linkers were attached, the secretion was significantly increased. Through this result, it could be seen that the negatively charged NKC increased the protein secretion.

### [Example 14] Increased Protein Secretion by Negative Supercharge

The present inventors have observed the tendency of protein secretion by replacing amino acids of proteins to negatively charged amino acids, to confirm secretion efficiency changes by changing the protein charge.

For this, negative charge supercharge −10 and positive charge supercharge+13 were produced from Streptavidin (SAV) wild type protein, and similar to this, supercharge proteins with the negative charger supercharge −20 and positive charge supercharge+19 were produced from glutathione S-transferase (GST), to analyze the protein secretion, and the result was shown in the following Table 16.

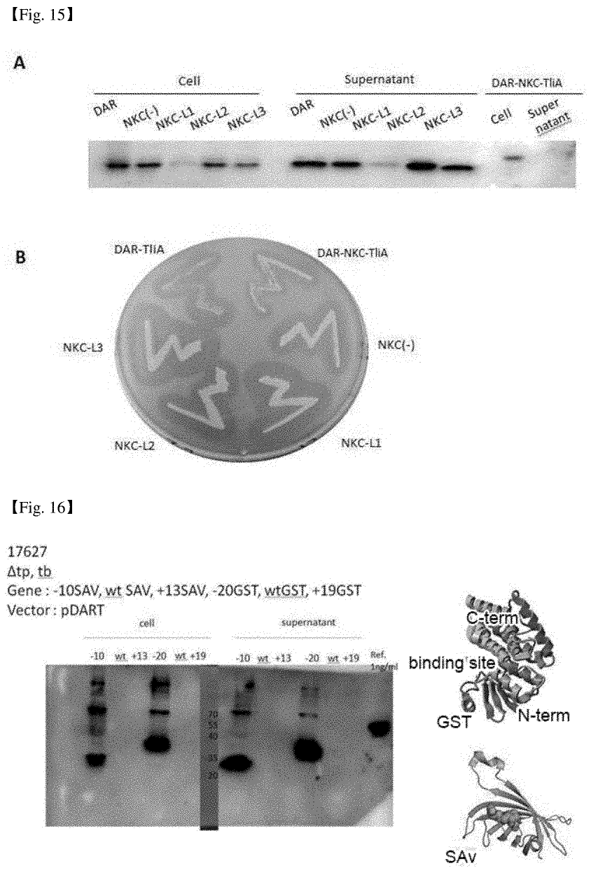

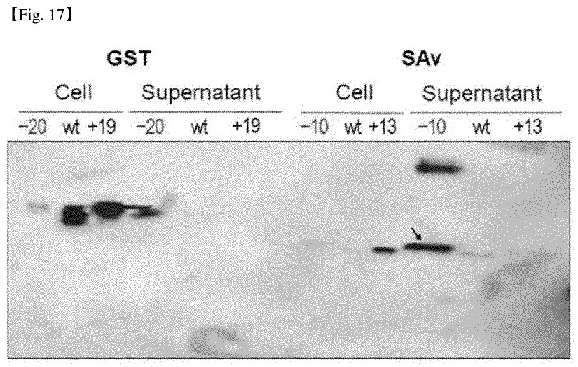

FIG. 16 represents the analysis result of secretion of −10SAV, wtSAV, +13SAV and −20GST, wtGST, and +19GST (SAV: streptavidin/GST: glutathione S-transferase). SAV (135aa) produces a tetramer and GST (215aa) produces a dimer. In gene synthesis, the charge of monomers was calculated (-10SAV: pI4.96/wtSAV:pI6.76/+13SAV: pI10.29/-20GST: pI4.73/wtGST: pI7.86/+19GST: pI9.87).

As shown in FIG. 16, it could be confirmed that negatively charged supercharger proteins were present in cells a lot and they were secreted well, but it could be seen that the wild type protein and positively charged supercharge proteins were not expressed and secreted, and it could be confirmed that the negative charge supercharge increased the protein secretion.

Similarly, while looking at the structure randomly without using AvNAPSA method, supercharged glutathione S-transferase (GST) and streptavidin (Say), in which protruding amino acids were replaced with aspartic acid or arginine, were added to pDART and were expressed, to perform western blotting, and the result was shown in FIG. 17.

As FIG. 17, it could be seen that the extracellular (Supernatant) to intracellular (Cell) protein concentration ratio of negatively supercharged proteins (indicated by red) was remarkably increased. On the other hand, positively supercharged proteins were detected in cells at a significantly high concentration, but they were not detected or were detected at a low concentration outside of cells (Supernatant).

### [Example 15] Confirmation of Extracellular Secretion Increase of Negatively Supercharged Protein Using AvNAPSA Method

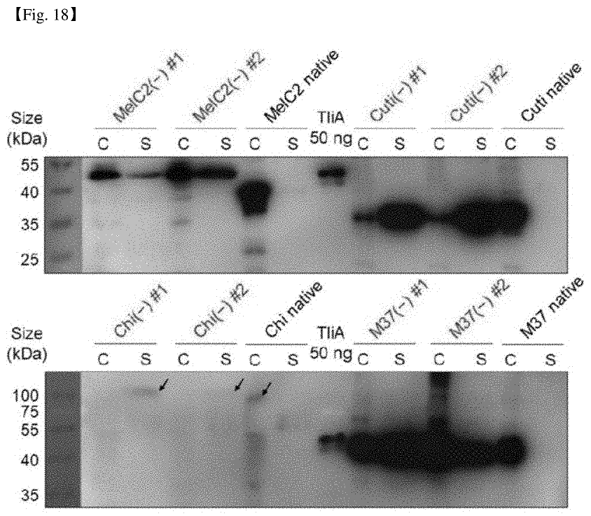

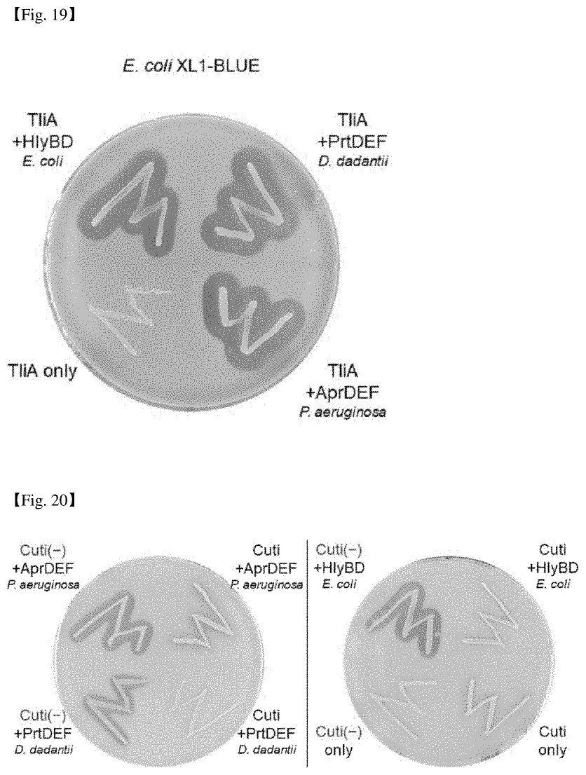

MelC₂ tyrosinase, cutinase (Cuti), Chitinase (Chi), and M37 lipase were negatively sugercharged by AvNAPSA method, and then negatively supercharged proteins (red) and non-supercharged natural proteins corresponding thereto (black) were added to pDART plasmids, respectively, and were expressed to detect them by western blotting, and the result was shown in FIG. 18.

Specifically, the negatively supercharging method using AvNAPSA is as follows. At first, arpartic acid and glutamic acid are replaced and enter to a suitable position to obtain the negatively supercharged protein sequence by AvNAPSA algorithm (1. Lawrence M S, Phillips K J, Liu D R. Supercharging Proteins Can Impart Unusual Resilience. Journal of the American Chemical Society 2007; 129: 10110-10112.). Then, the DNA sequence corresponding to the protein sequence was synthesized, and the synthesized DNA sequence was added to pDART plasmid and then negatively supercharged proteins were prepared.

It could be seen the negatively supercharged proteins were observed not only inside of cells (C) but also outside of cells (S) at a very high concentration, different from natural proteins which were not detected at all outside of cells, and their secretion were remarkably increased. In case of MelC2 tyrosinase protein, small sequence differences including His-tag result in small size differences between supercharged proteins and natural proteins.

In other words, through the experiment, the present inventors have confirmed that proteins which were not secreted in the past could be extracellularly secreted with considerable efficiency, by supercharging proteins such as tyrosinase, cutinase, and the like, to which the secretion production method was not applicable by conventional techniques, using AvNAPSA algorithm.

### [Example 16] Confirmation of TliA Protein Secretion in Cells in which T1SS Transporters Isolated from Three Different Kinds of Bacteria are Expressed

16-1. Escherichia coli HlyBD+TolC, Dickeya dadantii PrtDEF, Pseudomonas aeruginosa AprDEF Isolation

The present inventors amplified certain part of operon comprising HlyB, HlyD genes from isolated genome of Escherichia coli CFT073 strain (Genbank AE014075) through PCR using two primers of hlyBD-s (SEQ ID NO: 34: GGGGAGCTCGGATTCTTGTCATAAAATTGATT), hlyBD-a (SEQ ID NO: 35: GGGGGATCCTTAACGCTCATGTAAACTTTCT), and plasmid pSTV-HlyBD was prepared in which this was inserted in order together with start codon and kozak sequence to pSTV plasmid (one of derivatives of pACYC plasmid) by amplifying transporter genes from genome of each strain through PCR, respectively. TolC consisting of transporters together with HlyB and HlyC was not comprised separately, since it is produced by E. coli itself.

In addition, the present inventors prepared the plasmid expressing three genes of PrtD, PrtE and PrtF of Dickeya dadantii, pEcPrtDEF (Delepelaire P, Wandersman C Protein secretion in gram-negative bacteria. The extracellular metalloprotease B from Erwinia chrysanthemi contains a C-terminal secretion signal analogous to that of Escherichia coli alpha-hemolysin. J Biol Chem. 1990; 265:17118-17125) and the plasmid expressing three genes of AprD, AprE and AprF of Pseudomonas aeruginosa, pAGS8 (Duong F, Soscia C, Lazdunski A, Murgier M. The Pseudomonas fluorescens lipase has a C-terminal secretion signal and is secreted by a three-component bacterial ABC-exporter system. Mol Microbiol. 1994; 11:1117-1126).

16-2. Confirmation of Protein Secretion in Cells in which T1SS Transporters Isolated from Three Different Kinds of Bacteria are Expressed

The present inventors introduced one plasmid which the gene of TliA protein (original substrate of TliDEF transporter) was inserted to pQE184 plasmid, and one of plasmids expressing one kind of T1SS transporters isolated from three different kinds of bacteria prepared above (namely, pSTV-HlyBD expressing Escherichia coli HlyBD, pEcPrtDEF expressing Dickeya dadantii PrtDEF, pAGS8 expressing Pseudomonas aeruginosa AprDEF) to E. coli by heat shock method simultaneously and expressed TliA and one of three transporters simultaneously, and then measured secretion of the recombinant target proteins from lipase enzyme activity measuring media to outside of cells through color changes of colony peripheral media, and the result was shown in FIG. 19.

As shown in FIG. 19, it could be confirmed that all the three transporters of Escherichia coli HlyBD+TolC (E. coli expresses the original TolC protein), Dickeya dadantii PrtDEF, and Pseudomonas aeruginosa AprDEF secreted TliA protein successfully. This can be inferred from the fact that halo is not observed in the strain in which only TliA protein is expressed (TliA only) without further expression of transporter proteins in E. coli. The result means that T1SS proteins of Escherichia coli, Dickeya dadantii, and Pseudomonas aeruginosa other than Pseudomonas fluorescens can recognize the LARD3 signal sequence of TliA.

### [Example 17] Confirmation of Cutinase Protein Secretion in Cells in which T1SS Transporters Isolated from Three Different Kinds of Bacteria are Expressed

17-1. Preparation of Negatively Supercharged Cutinase Protein

Negatively supercharged cutinase protein (Cuti(-)) was prepared using AvNAPSA method to cutinase protein (Cuti).

17-2. Confirmation of Cutinase Protein Secretion in Cells in which T1SS Transporters Isolated from Three Different Kinds of Bacteria are Expressed

After attaching the LARD3 signal sequence by the method of inserting cutinase genes to pLARD3 plasmid in which the gene of LARD3 signal sequence was inserted right behind of the multiple cloning site, based on pUC19 plasmid, to cutinase protein and negatively supercharged cutinase protein, with the plasmid expressing three different kinds of T1SS transporter proteins (Escherichia coli HlyBD+TolC, Dickeya dadantii PrtDEF, Pseudomonas aeruginosa AprDEF) obtained by the method of Example 16, similar to the method of Example 16, two plasmids were introduced to E. coli cells simultaneously and were expressed simultaneously, and they were culture in cutinase enzyme activity measuring media at 37° C. for 3 days, and then the protein secretion to outside of E. coli was measured through color changes of colony peripheral media, and the result was shown in FIG. 20.

As shown in FIG. 20, it could be observed that the secretion of negatively supercharged cutinase was remarkably high than non-negatively-supercharged cutinase, in all the three kinds of T1SS transporter proteins. In the same manner, it could be inferred through comparison to control groups in which an empty plasmid was added instead of the transporter plasmid (Cuti(-) only, Cuti only).

### [Example 18] Confirmation of Extracellular Secretion of Cutinase Protein Using Western Blotting

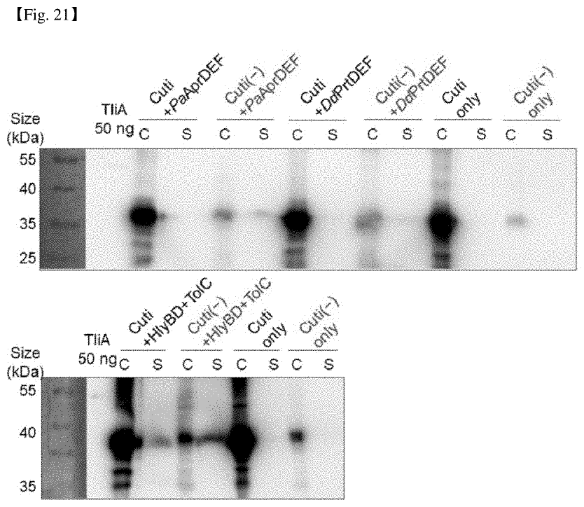

After attaching the LARD3 signal sequence to cutinase protein (Cuti) and negatively supercharged cutinase protein (Cuti(-)), they were expressed in E. coli with three different kinds of T1SS transporter proteins obtained by the method of Example 16 and were liquid cultured, and then the intracellular and extracellular protein concentration was detected by western blotting, and the result was shown in FIG. 21.

As shown in FIG. 21, it could be observed that the secretion of negatively supercharged cutinase was remarkably high than non-negatively-supercharged cutinase, in all the three kinds of T1SS transporter proteins. In the same manner, the secretion fact could be inferred by comparison to control groups in which an empty plasmid was added instead of the transporter plasmid (Cuti(-) only, Cuti only).

### [Example 19] Confirmation of Extracellular Secretion of M37 Lipase Protein Using Western Blotting

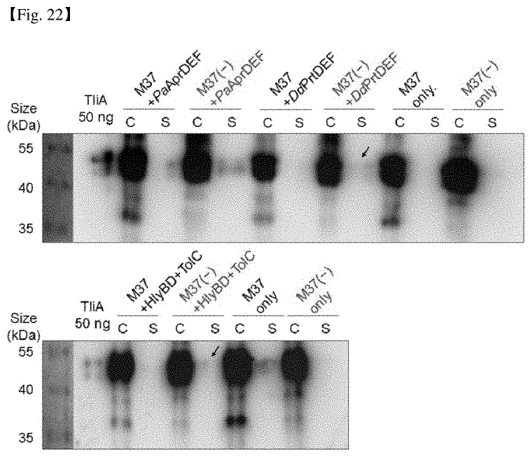

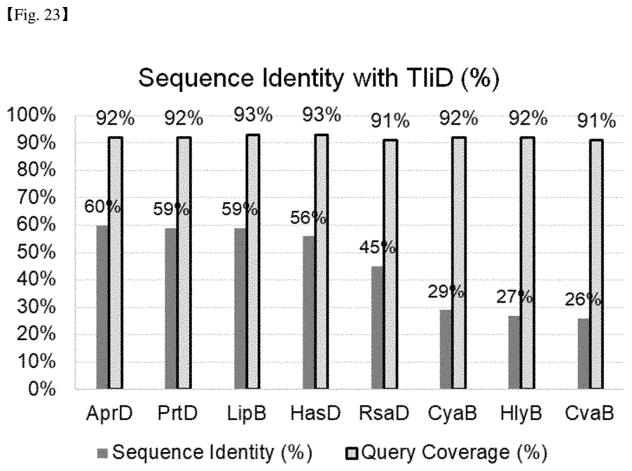

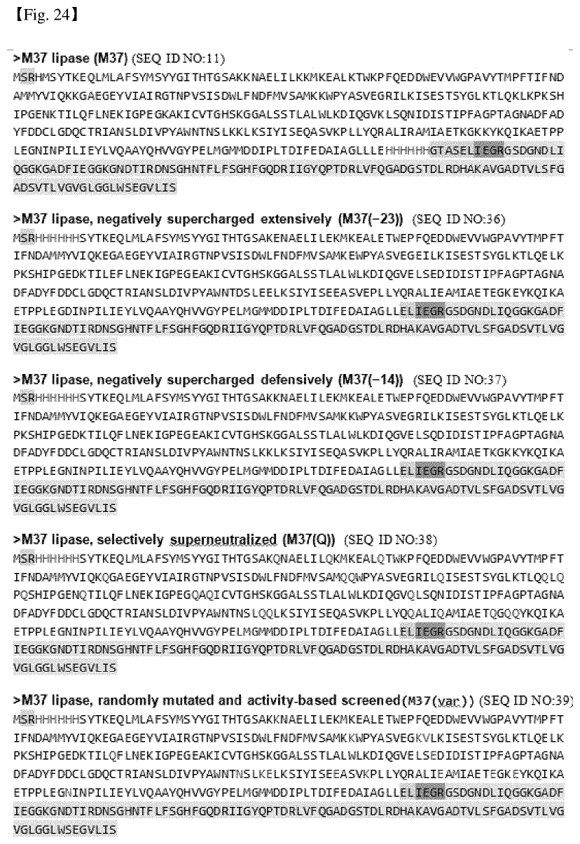

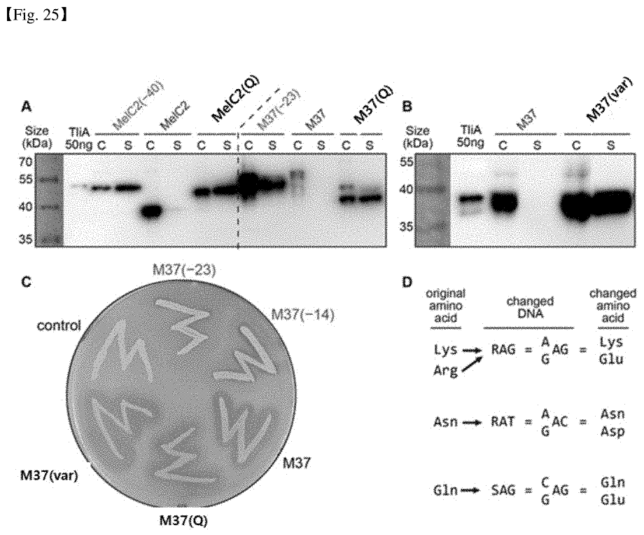

After attaching the LARD3 signal sequence to M37 lipase protein and negatively supercharged M37 lipase protein (M37(-)), they were expressed in E. coli with three different kinds of T1SS transporter proteins obtained by the method of Example 16 and were liquid cultured, and then the intracellular and extracellular protein concentration was detected by western blotting, and the result was shown in FIG. 22.

As shown in FIG. 22, it could be observed that the secretion of negatively supercharged M37 was remarkably high than non-negatively-supercharged M37, in all the three kinds of T1SS transporter proteins. In the same manner, the secretion fact could be inferred by comparison to control groups in which an empty plasmid was added instead of the transporter plasmid (M37(-) only, M37 only).

### [Example 20] Evaluation of Sequence Identity of T1SS ABC Transporters

The sequence identity of TliD of TliDET transporter of Pseudomonas fluorescens and T1SS ABC transporters of Escherichia coli HlyBD+TolC, Dickeya dadantii PrtDEF, and Pseudomonas aeruginosa AprDEF with ABC proteins of other kinds of T1SS transporters was measured, and the result was shown in FIG. 23. FIG. 23 represents the sequence identity between TliDEF transporter and various T1SS transporters, and the proportion occupied by the sequence-like portion in the entire sequence.

Specifically, the sequence identity between transporter proteins was calculated using NCBI BLASTp algorithm, and the indicated sequence identity was calculated by omitting some sequences that greatly differ from each other according to the normal calculation method of the algorithm, and limiting within the query coverage. As a result, the omitted sequence portion was less than 10% in any case, suggesting that the sequence identity was very reliable.

The sequence identity of TilD of TliDEF transporter with various T1SS ABC transporters varied from relatively high to relatively low. Among them, the sequence identity of three T1SS transporters of AprD, PrtD, and HlyB which were examples in Examples 16, 17, 18 and 19 were exhibited as 60%, 59%, and 27%, respectively.

Accordingly, the present inventors confirmed that the protein secretion enhancement technology of negatively supercharging was not limited to Pseudomonas fluorescens microorganism TliDEF transporters, and could be widely applied to various T1SS transporters having about 27% of the amino acid sequence identity (homology).

### [Example 21] the Preparation of the Target Protein with Lowered pI by Substituting with Neutral Amino Acids

As described above, negatively supercharging the target protein to enhance the secretion came along with the problem of losing the enzymatic activity in some cases. To deal with this problem, The inventors devised a method to make the substitution less damaging to the protein's overall fold, which was replacing these solvent-exposed positively charged residues to the neutral hydrophilic amino acids, not negatively charged ones. The inventors primarily used relatively bulky glutamine to replace arginine and lysine, as both of them were quite bulky amino acids. This way, the genes encoding the two proteins MelC2(Q) and M37(Q) was prepared. The “(Q)” parts designate that the solvent-exposed amino acids were replaced with glutamine (single-letter code Q).

Further on, the inventors refer to the technique as “Superneutralization” or “Superneutralizing” which is a process of removing charges by replacing charged residues on the solvent-exposed surface of a protein or protein complexes with neutrally-charged amino acids. The term “selective superneutralization of positive charges” is used to describe a process of superneutralization applied specifically and solely on positively charged residues so that the mutated protein mainly consists of negative charge. It turned out that removing positive charges via superneutralization (not adding any additional negative charge) also improves the secretion of proteins dramatically, in both MelC2 and M37 (Error! Reference source not found.A). Besides, the superneutralized M37(Q) exhibited enzymatic activity, as can be seen from the plate activity assay (Error! Reference source not found.C).

More specifically, in FIG. 23A, the solvent-accessible positively charged residues were replaced with glutamine (single-letter code Q), a neutral hydrophilic amino acid. The two resultant proteins, MelC2(Q) and M37(Q) were highly localized to the culture supernatant, compared to their wild-types. The negatively supercharged versions of these two proteins, MelC2(-40) and M37(-23) were also loaded as a comparison.

In FIG. 23C, the colonies of P. fluorescens cells expressing the M37 derivatives were streaked on the LB agar plate supplemented with 0.5% colloidal glyceryl tributyrate. The lipase activity of the secreted M37 lipase creates a visible clear halo around the streaks. As a negative control, P. fluorescens cells harboring pDART-GFP plasmid was streaked as well. Negatively supercharged M37 variants, M37(-23) and M37(-14) had significantly lower halo size compared to the wild-type M37. Especially, M37(-23) exhibited little enzymatic activity even though it was mainly localized in the extracellular space. On the other hand, however, M37(Q) and M37(var) had large halo size, comparable or even larger than the wild-type.

The inventors did not test the activity of MelC2(Q), mainly because the MelC2 protein itself is generally not active without its caddie protein MelC1, which is not present in the P. fluorescens expression host when introduced via pDART plasmid. It might be possible to examine the activity of pDART-inserted MelC2(Q) by co-expressing MelC1 in P. fluorescens, but that experiment was not performed here. Comprehending the results, the superneutralization approach was proven to be a better option than the conventional supercharging method in terms of the preservation of protein activity, while being just as effective in terms of the secretion enhancement.

Specifically, the computational designing of supercharged or superneuturalized proteins were performed. The inventors utilized the AvNAPSA algorithm to boost the productivity and reproducibility of supercharged protein designing. The AvNAPSA method, an abbreviation for Average Neighbor Atoms per Sidechain Atom was developed by Liu group to automatically design supercharged proteins, in their pursuit of making a resilient folded protein and for animal cellular protein targeting. In this paper, however, the supercharging protocol is used to generate proteins that are compatible with ABC transporter secretion. AvNAPSA algorithm automatically scores the residues according to the exposure to the external space, rather than the facing other parts of the protein. More specifically, it calculates the number of atoms within a certain distance from each atom of the side chain and returns the AvNAPSA score for each residue. The lower the score, the more exposed the residue to the solvent. We gradually mutated positively charged residues in the increasing order of AvNAPSA score until the level of the mutation was similar to the level of mutation we would have if we performed manual supercharging. The exact AvNAPSA thresholds for our mutated proteins are given in Table 5. Also note that the inventors excluded any residue proximal (closer than seven residues apart) to the active site residues.

TABLE 5

| | Full name | | |
|---|---|---|---|
| Abbrev. | (SEQ ID NO) | Source | Source type |
| M37 | M37 lipase | Photobacterium lipolyticum | Genomic DNA |
| | (SEQ ID NO: 11) | | |
| M37(−23) | M37, −23 negatively | AvNAPSA supercharging, | Synthesized |
| | supercharged | threshold = 100 | |
| | (SEQ ID NO: 36) | | |
| M37(−14) | M37 lipase, −14 negatively | AvNAPSA supercharging, | Synthesized |
| | supercharged | threshold = 90 | |
| | (SEQ ID NO: 37) | | |
| M37(Q) | M37, selectively | Manually superneutralized | Synthesized |
| | superneutralized | | |
| | (SEQ ID NO: 38) | | |
| M37(var) | M37, randomly mutated | Random mutation and activity | Synthesized via |
| | and screened | screening | mixed-base |
| | (SEQ ID NO: 39) | | |

Synthesizing and Cloning the Gene of Variationally Supercharged M37

The inventor prepared the DNA sequence of variationally supercharged M37 lipase, which we named M37(var), by replacing the codons for the surface-exposed positively charged amino acid residues with the degenerate codon. For example, the IUPAC DNA code R denotes the purine base, which is guanine or adenine. The inventor aimed to find the Goldilocks variant somewhere between M37(-14), which had enzymatic activity but was not secreted, and M37(-23), which was secreted but had no enzymatic activity. The inventor examined the amino acid sequences of M37(-23) and M37(-14) and marked the residues where the two differed. Then, the inventors replaced codons for those residues with the degenerate codons. For instance, the residue Lys36 of M37 lipase was replaced by glutamic acid in M37(-23) but remained unchanged in M37(-14). Therefore, the inventor placed the degenerate codon “RAG” at that position. For the degenerate codon RAG, there were two possible outcomes: GAG, which codes for glutamic acid; and AAG, which codes for lysine. The inventors would have preferred to use the set “glutamic acid or arginine”, but such a combination was impossible. Therefore, the inventors used the RAG codon in place of these residues as well. The exact sequence we ordered for the construction of M37(var) is given in Supplementary Section A. After the DNA sequence was designed, the inventor used the DNAWorks web server (https://hpcwebapps.cit.nih.gov/dnaworks/) to convert the sequence into a set of synthesizable primers. The used parameters were as the following: oligo length 58 nucleotides, annealing temperature 62° C., oligo concentration 1.00×10⁻⁷ M, Na⁺/K⁺ concentration 0.05 M, Mg²⁺ concentration 0.002 M, number of solutions 1, no “TBIO” mode (“PTDS” mode was used instead). Then, the inventors manually examined the output oligos and made sure that no degenerate codon was present at the end of any overlapping region between oligos. The inventors ordered the oligos from Cosmogenetech, and assembled them using the PCR-based DNA synthesis method described in a previous publication. The obtained PCR product was purified, restricted, and then introduced into pDART plasmid like the rest of the genes handled in the example.

### [Example 22] the Preparation of the Target Protein with Lowered pI by Mutation and Activity Screening Method

Each residue modification induces some change in the protein's 3-dimensional structure, but the amount of the change varies. Some residue mutations may barely affect the overall structure except for its side chain itself, while others could accompany a serious distortion in the secondary and/or tertiary structure. However, we cannot yet accurately evaluate the magnitude of structural change induced by each point mutation, since we are mutating multiple residues at once. So, our idea was to leave the evaluation to the cells, not the human researchers. We synthesized the gene of M37 lipase, with the random mutations. Specifically, the solvent-exposed positively charged amino acids (arginine and lysine) were randomly mutated into negatively charged or neutral hydrophilic amino acids. We used mixed-base DNA synthesis to randomly choose codons between the two amino acids. After the randomly mutated genes were synthesized, they were incorporated into the pDART plasmid, and then introduced into E. coli. We screened for the clone that exhibited the most prominent halo in the lipase activity assay LB agar plate. The resulting clone was then characterized via DNA sequencing. The resulting clone had both excellent secretion (Error! Reference source not found.B) and activity (Error! Reference source not found.C). The mixed-base strategy utilized to prepare M37(var) is given in Error! Reference source not found.D.

More specifically, in FIG. 23B, the solvent-accessible positively charged or neutral hydrophilic residues of M37 lipase were randomly mutated into neutral or negatively charged amino acids. Then, the clone which exhibited the largest halo on the activity plate assay was selected and sequenced. The inventors named this mutant lipase “M37(var)”. The inventors analyzed the secretion of M37(var) via western blot. It turned out that it was indeed secreted well. FIG. 23D shows the mixed-based codon strategy utilized to prepare M37(var) mutant.

1. An expression vector for extracellular secretion of a target protein in bacteria, comprising an expression cassette including a nucleotide sequence encoding Lipase ABC transporter recognition domain (LARD) and a nucleotide sequence encoding a target protein which are operably linked, wherein the LARD and the target protein have acidic pI and is expressed as a fusion protein.

2. The expression vector of claim 1, wherein the expression vector comprises a nucleotide sequence comprising ABC transporter of bacterial Type 1 Secretion System (T1SS).

3. The expression vector of claim 1, wherein the bacteria comprises an ABC transporter of Type 1 Secretion System (T1SS).

4. The expression vector of claim 2, wherein the ABC transporter of T1SS is a transporter having at least 20% of nucleotide sequence identify with TliDEF transporter of Pseudomonas fluorescence.

5. The expression vector of claim 1, wherein the bacteria is at least one Gram-negative bacteria selected from the group consisting of Pseudomonas sp., Dickeya sp., and Escherichia sp.

6. The expression vector of claim 2, wherein the ABC transporter of T1SS is LipBCD of Serratia marcescens, HasDEF of Serratia marcescens, CyaBDE of Bordetella pertussis, CvaBA+TolC of Escherichia coli, RsaDEF of Caulobacter crescentus, Pseudomonas aeruginosa AprDEF (PaAprDEF), Dickeya dadantii PrtDEF (DdPrtDEF), or Escherichia coli HlyBD+TolC.

7. The expression vector of claim 3, wherein the ABC transporter of T1SS is LipBCD of Serratia marcescens, HasDEF of Serratia marcescens, CyaBDE of Bordetella pertussis, CvaBA+TolC of Escherichia coli, RsaDEF of Caulobacter crescentus, Pseudomonas aeruginosa AprDEF (PaAprDEF), Dickeya dadantii PrtDEF (DdPrtDEF),or Escherichia coli HlyBD+TolCi.

8. The expression vector of claim 1, wherein the nucleotide sequence encoding a target protein further comprises a nucleotide sequence encoding an acidic peptide consisting of 6 to 20 amino acids.

9. The expression vector of claim 1, wherein the target protein is a mutated protein with lowered pI value obtained by deleting at least one of the basic amino acids in the target protein, or by substituting them with other amino acids.

10. The expression vector of claim 9, wherein at least one of the basic amino acids in the target protein is substituted with at least one amino acid selected from the group consisting of acidic amino acids and neutral amino acids.

11. The expression vector of claim 9, wherein the other amino acids is at least one amino acid selected from the group consisting of aspartic acid, glutamic acid, and glutamine.

12. The expression vector of claim 1, wherein the target protein is negatively supercharged protein obtained by performing the following steps:
selecting at least an amino acid not affecting the structure of target protein by having a functional group protruding in three dimensional structure of the target protein, and
substituting the selected amino acid with at least one selected from the group consisting of acidic amino acids and neutral amino acids, when the selected amino acid is basic.

13. The expression vector of claim 1, wherein the target protein is negatively supercharged protein obtained by performing the following steps:
selecting at least an amino acid not affecting the structure of target protein by having a functional group protruding in three dimensional structure of the target protein,
mutating at least one selected amino acid, into at least one selected from neutral amino acids and acidic amino acids to produce mutated target protein, and
selecting the mutated target protein having activity.

14. A cell comprising an expression vector according to claim 1.

15. A method of performing an extracellular secretion of a target protein in a bacterial cell, comprising:
obtaining a target protein with lowered pI by deleting at least one basic amino acid in the target protein, or by substituting them with other amino acids,
preparing an expression cassette including a nucleotide sequence encoding Lipase ABC transporter recognition domain (LARD) and a nucleotide sequence encoding a target protein which are operably linked, wherein the LARD and the target protein have acidic pI and is expressed as a fusion protein, and
expressing the expression cassette in the bacterial cell.

16. The method of claim 15, wherein the bacterial cell further comprises an ABC transporter of Type 1 Secretion System (T1SS), or an expression cassette comprising a nucleotide sequence encoding ABC transporter of bacterial Type 1 Secretion System (T1SS).

17. The method of claim 15, wherein at least one of the basic amino acids in the target protein is substituted with at least one amino acid selected from the group consisting of acidic amino acids and neutral amino acids.

18. The method of claim 15, wherein the other amino acids is at least one amino acids selected from the group consisting of aspartic acid, glutamic acid, and glutamine.

19. The method of claim 15, wherein the target protein with lowered pI is negatively supercharged protein obtained by performing the following steps:
selecting at least an amino acid not affecting the structure of target protein by having a functional group protruding in three dimensional structure of the target protein, and
substituting the selected amino acid with at least one selected from the group consisting of acidic amino acids and neutral amino acids, when the selected amino acid is basic.

20. The expression vector of claim 1, wherein the target protein is negatively supercharged protein obtained by performing the following steps:
selecting at least an amino acid not affecting the structure of target protein by having a functional group protruding in three dimensional structure of the target protein,
mutating at least one selected amino acid, into at least one selected from neutral amino acids and acidic amino acids to produce mutated target protein, and
selecting the mutated target protein having activity.


SEQUENCE LISTING (39 sequences)

SEQ ID NO: 1 - protein, 476 aa, Artificial Sequence
  Amino acid sequence of TliA wild type
MGVFDYKNLGTEASKTLFADATAITLYTYHNLDNGFAVGYQQHGLGLGLPATLVGALLGSTDSQGVIPGIPWNPDSEKAALDAVHAAGWTPISASALGYGGKVDARGTFFGEKAGYTTAQAEVLGKYDDAGKLLEIGIGFRGTSGPRESLITDSIGDLVSDLLAALGPKDYAKNYAGEAFGGLLKTVADYAGAHGLSGKDVLVSGHSLGGLAVNSMADLSTSKWAGFYKDANYLAYASPTQSAGDKVLNIGYENDPVFRALDGSTFNLSSLGVHDKAHESTTDNIVSFNDHYASTLWNVLPFSIANLSTWVSHLPSAYGDGMTRVLESGFYEQMTRDSTIIVANLSDPARANTWVQDLNRNAEPHTGNTFIIGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 2 - protein, 589 aa, Artificial Sequence
  Amino acid sequence of TliA expressed in pDART plasmid
MSRMGVFDYKNLGTEASKTLFADATAITLYTYHNLDNGFAVGYQQHGLGLGLPATLVGALLGSTDSQGVIPGIPWNPDSEKAALDAVHAAGWTPISASALGYGGKVDARGTFFGEKAGYTTAQAEVLGKYDDAGKLLEIGIGFRGTSGPRESLITDSIGDLVSDLLAALGPKDYAKNYAGEAFGGLLKTVADYAGAHGLSGKDVLVSGHSLGGLAVNSMADLSTSKWAGFYKDANYLAYASPTQSAGDKVLNIGYENDPVFRALDGSTFNLSSLGVHDKAHESTTDNIVSFNDHYASTLWNVLPFSIANLSTWVSHLPSAYGDGMTRVLESGFYEQMTRDSTIIVANLSDPARANTWVQDLNRNAEPHTGNTFIIGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLISELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 3 - protein, 614 aa, Artificial Sequence
  Amino acid sequence of NKC-TliA
MSRHMGTAPKAMKLLKKLLKLQKKGIGSMGVFDYKNLGTEASKTLFADATAITLYTYHNLDNGFAVGYQQHGLGLGLPATLVGALLGSTDSQGVIPGIPWNPDSEKAALDAVHAAGWTPISASALGYGGKVDARGTFFGEKAGYTTAQAEVLGKYDDAGKLLEIGIGFRGTSGPRESLITDSIGDLVSDLLAALGPKDYAKNYAGEAFGGLLKTVADYAGAHGLSGKDVLVSGHSLGGLAVNSMADLSTSKWAGFYKDANYLAYASPTQSAGDKVLNIGYENDPVFRALDGSTFNLSSLGVHDKAHESTTDNIVSFNDHYASTLWNVLPFSIANLSTWVSHLPSAYGDGMTRVLESGFYEQMTRDSTIIVANLSDPARANTWVQDLNRNAEPHTGNTFIIGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLISELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 4 - protein, 644 aa, Artificial Sequence
  Amino acid sequence of CTP-TliA
MSRMRGSHHHHHHGMASMTGGQQMGRDLYDDDDKDRWGSMYGRRARRRRRRSMAGTGGMGVFDYKNLGTEASKTLFADATAITLYTYHNLDNGFAVGYQQHGLGLGLPATLVGALLGSTDSQGVIPGIPWNPDSEKAALDAVHAAGWTPISASALGYGGKVDARGTFFGEKAGYTTAQAEVLGKYDDAGKLLEIGIGFRGTSGPRESLITDSIGDLVSDLLAALGPKDYAKNYAGEAFGGLLKTVADYAGAHGLSGKDVLVSGHSLGGLAVNSMADLSTSKWAGFYKDANYLAYASPTQSAGDKVLNIGYENDPVFRALDGSTFNLSSLGVHDKAHESTTDNIVSFNDHYASTLWNVLPFSIANLSTWVSHLPSAYGDGMTRVLESGFYEQMTRDSTIIVANLSDPARANTWVQDLNRNAEPHTGNTFIIGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLISELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 5 - protein, 454 aa, Artificial Sequence
  Amino acid sequence of Mannanase
MSRHHHHHHTVSPVNPNAQQTTKAVMNWLAHLPNRTENRVLSGAFGGYSHDTFSMAEADRIRSATGQSPAIYGCDYARGWLETANIEDSIDVSCNSDLMSYWKNDGIPQISLHLANPAFQSGHFKTPITNDQYKKILDSSTAEGKRLNTMLSKIADGLQELENQGVPVLFRPLHEMNGERFWWGLTSYNQKDNERISLYKQLYKKIYHYMTDTRGLDHLIWVYSPDANRDFKTDFYPGASYVDIVGLDAYFQDAYSINGYDQLTALNKPFAFTEVGPQTANGSFDYSLFINAIKHRYPKTIYFLAWNDEWSPAVNKGASALYHDSWTLNKGEIWNGDSLTPIVEELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 6 - protein, 266 aa, Artificial Sequence
  Amino acid sequence of Mussel adhesion protein
MSSMRGSHHHHHHGSASAKPSYPPTYKAKPSYPPTYKAKPSYPPTYKGCSSEEYKGGYYPGNSNHYHSGGSYHGSGYHGGYKGKYYGKAKKYYYKYKNSGKYKYLKKARKYHRKGYKKYYGGSSEFAKPSYPPTYKAKPSYPPTYKAKPSYPPTYKELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 7 - protein, 483 aa, Artificial Sequence
  Amino acid sequence of Maltose binding protein
MSRKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITKELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 8 - protein, 240 aa, Artificial Sequence
  Amino acid sequence of Thioredoxin
MSRMLHQQRNQHARLIPVELYMSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 9 - protein, 335 aa, Artificial Sequence
  Amino acid sequence of Cutinase
MSRHHHHHHAPTSNPAQELEARQLGRTTRDDLINGNSASCADVIFIYARGSTETGNLGTLGPSIASNLESAFGKDGVWIQGVGGAYRATLGDNALPRGTSSAAIREMLGLFQQANTKCPDATLIAGGYSQGAALAAASIEDLDSAIRDKIAGTVLFGYTKNLQNRGRIPNYPADRTKVFCNTGDLVCTGSLIVAAPHLAYGPDARGPAPEFLIEKVRAVRGSALEELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 10 - protein, 762 aa, Artificial Sequence
  Amino acid sequence of Chitinase
MSRHHHHHHANSPKQSQKIVGYFPSWGVYGRNYQVADIDASKLTHLNYAFADICWNGKHGNPSTHPDNPNKQTWNCKESGVPLQNKEVPNGTLVLGEPWADVTKSYPGSGTTWEDCDKYARCGNFGELKRLKAKYPHLKTIISVGGWTWSNRFSDMAADEKTRKVFAESTVAFLRAYGFDGVDLDWEYPGVETIPGGSYRPEDKQNFTLLLQDVRNALNKAGAEDGKQYLLTIASGASRRYADHTELKKISQILDWINIMTYDFHGGWEATSNHNAALYKDPNDPAANTNFYVDGAINVYTNEGVPVDKLVLGVPFYGRGWKSCGKENNGQYQPCKPGSDGKLASKGTWDDYSTGDTGVYDYGDLAANYVNKNGFVRYWNDTAKVPYLYNATTGTFISYDDNESMKYKTDSIKTKGLSGAMFWELSGDCRTSPKYSCSGPKLLDTLVKELLGGPINQKDTEPPTNVKNIVVTNKNSNSVQLNWTASTDNVGVTEYEITAGEEKWSTTTNSITIKNLKPNTEYKFSIIAKDAAGNKSQPTALTVKTDEANMTPPDGNGTATFSVTSNWGSGYNFSIIIKNNGTNPIKNWKLEFDYSGNLTQVWDSKISSKTNNHYVITNAGWNGEIPPGGSITIGGAGTGNPAELLNAVISENELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 11 - protein, 466 aa, Artificial Sequence
  Amino acid sequence of M37 lipase
MSRHMSYTKEQLMLAFSYMSYYGITHTGSAKKNAELILKKMKEALKTWKPFQEDDWEVVWGPAVYTMPFTIFNDAMMYVIQKKGAEGEYVIAIRGTNPVSISDWLFNDFMVSAMKKWPYASVEGRILKISESTSYGLKTLQKLKPKSHIPGENKTILQFLNEKIGPEGKAKICVTGHSKGGALSSTLALWLKDIQGVKLSQNIDISTIPFAGPTAGNADFADYFDDCLGDQCTRIANSLDIVPYAWNTNSLKKLKSIYISEQASVKPLLYQRALIRAMIAETKGKKYKQIKAETPPLEGNINPILIEYLVQAAYQHVVGYPELMGMMDDIPLTDIFEDAIAGLLLEHHHHHHGTASELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 12 - protein, 505 aa, Artificial Sequence
  Amino acid sequence of Capsid
MSRMARKKSTPRRRKAVKRRRTVRRRQSPKARVRSTTTKAKRRISPSGSGSQHLTVRKQPFSNATSQPKILDGALTSSLSRRLQNVIGLTNGNGGLGTEIMHIFFAPTLGIPLIAMNSAEGVALRPSSSADPFFIGFPGQTIKFDYVSSGTTPPATGNLVTFSNECGFSKWRIVSQGLRMELANSDEENDGWFEAVRFNWRNNPADICFTPIDGTLGGAKTTDFAVAPSPVGMYALKDMAMVEQPGYTTGLLKDLKNHEFMLHPQSTTHDPIILEQSYEGTIGTTAADDMYYSVTSEVFELGNTVRGNTMKNSLVDNNMDWIYLRLHCRTNNGTTSNGSKLIVNAIQNLEVSFNPSSDFAAFQTINKMHPQQKKVDDQLNNSAEASNKRQKTGGGELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 13 - protein, 493 aa, Artificial Sequence
  Amino acid sequence of DnaJ
MSRMAKQDYYEILGVSKTAEEREIRKAYKRLAMKYHPDRNQGDKEAEAKFKEIKEAYEVLTDSQKRAAYDQYGHAAFEQGGMGGGGFGGGADFSDIFGDVFGDIFGGGRGRQRAARGADLRYNMELTLEEAVRGVTKEIRIPTLEECDVCHGSGAKPGTQPQTCPTCHGSGQVQMRQGFFAVQQTCPHCQGRGTLIKDPCNKCHGHGRVERSKTLSVKIPAGVDTGDRIRLAGEGEAGEHGAPAGDLYVQVQVKQHPIFEREGNNLYCEVPINFAMAALGGEIEVPTLDGRVKLKVPGETQTGKLFRMRGKGVKSVRGGAQGDLLCRVVVETPVGLNERQKQLLQELQESFGGPTGEHNSPRSKSFFDGVKKFFDDLTRGTASELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 14 - protein, 348 aa, Artificial Sequence
  Amino acid sequence of Eg15
MSRHHHHHHYKATTTRYYDGQEGACGCGSSSGAFPWQLGIGNGVYTAAGSQALFDTAGASWCGAGCGKCYQLTSTGQAPCSSCGTGGAAGQSIIVMVTNLCPNNGNAQWCPVVGGTNQYGYSYHFDIMAQNEIFGDNVVVDFEPIACPGQAASDWGTCLCVGQQETDPTPVLGNDTGSTPPGSSPPATSSSPPSGGGQQTLYGQCGGAGWTGPTTCQAPGTCKVQNQWYSQCLPGTASELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 15 - protein, 347 aa, Artificial Sequence
  Amino acid sequence of GFP
MSRMSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 16 - protein, 361 aa, Artificial Sequence
  Amino acid sequence of -30 Negatively supercharged GFP
MSRMGHHHHHHGGASKGEELFDGVVPILVELDGDVNGHEFSVRGEGEGDATEGELTLKFICTTGELPVPWPTLVTTLTYGVQCFSDYPDHMDQHDFFKSAMPEGYVQERTISFKDDGTYKTRAEVKFEGDTLVNRIELKGIDFKEDGNILGHKLEYNFNSHDVYITADKQENGIKAEFEIRHNVEDGSVQLADHYQQNTPIGDGPVLLPDDHYLSTESALSKDPNEDRDHMVLLEFVTAAGIDHGMDELYKELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 17 - protein, 361 aa, Artificial Sequence
  Amino acid sequence of +36 Positively supercharged GFP
MSRMGHHHHHHGGASKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYKELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 18 - protein, 164 aa, Artificial Sequence
  Amino acid sequence of epidermal growth factor
MNSDSECPLSHDGYCLHDGVCMYIEALDKYACNCVVGYIGERCQYRDLKWWELRSRIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 19 - protein, 559 aa, Artificial Sequence
  Amino acid sequence of Alkaline phosphatase
MSSMPVLENRAAQGDITAPGGARRLTGDQTAALRDSLSDKPAKNIILLIGDGMGDSEITAARNYAEGAGGFFKGIDALPLTGQYTHYALNKKTGKPDYVTDSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLKELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 20 - protein, 440 aa, Artificial Sequence
  Amino acid sequence of Phospholipase A1
MSMSLSFTSAIAPAAIQPPMVRTQPEPLSSSQPVEASATKAPVATLSQNSLNAQSLLNTLVSEISAAAPAAANQGVTRGQQPQKGDYTLALLAKDVYSTGSQGVEGFNRLSADALLGAGIDPASLQDAASGFQAGIYTDNQQYVLAFAGTNDMRDWLSNVRQATGYDDVQYNQAVSLAKSAKAAFGDALVIAGHSLGGGLAATAALATGTVAVTFNAAGVSDYTLNRMGIDPAAAKQDAQAGGIRRYSEQYDMLTGTQESTSLIPDAIGHKITLANNDTLSGIDDWRPSKHLDRSLTAHGIDKVISSMAEQKPWEAMANAHHHHHHGTASELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 21 - protein, 4 aa, Artificial Sequence
  Amino acid sequence of Factor Xa
IEGR

SEQ ID NO: 22 - protein, 104 aa, Artificial Sequence
  Amino acid sequence of LARD3 signal peptide
GSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 23 - protein, 119 aa, Artificial Sequence
  Amino acid sequence of pDART Translation Structure
MSRHMGTASELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 24 - protein, 131 aa, Artificial Sequence
  Amino acid sequence of pFD10 Translation Structure
MSSDDDDDDDDDDSRHMGTASELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 25 - protein, 130 aa, Artificial Sequence
  Amino acid sequence of pBD10 Translation Structure
MSRHMGTASELDDDDDDDDDDDIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 26 - protein, 130 aa, Artificial Sequence
  Amino acid sequence of pBE10 Translation Structure
MSRHMGTASELEEEEEEEEEEGIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 27 - protein, 130 aa, Artificial Sequence
  Amino acid sequence of pBH10 Translation Structure
MSRHMGTASELHHHHHHHHHHGIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 28 - protein, 130 aa, Artificial Sequence
  Amino acid sequence of pBR10 Translation Structure
MSRHMGTASELRRRRRRRRRRGIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 29 - protein, 16 aa, Artificial Sequence
  Amino acid sequence of LARD purification
VLSFGADSVTLVGVGL

SEQ ID NO: 30 - protein, 44 aa, Artificial Sequence
  Amino acid sequence of LARD secretion signal peptide
GSDGNDLIQGGKGADFIEGGKGNDTIRDNSGHNTFLFSGHFGQD

SEQ ID NO: 31 - protein, 174 aa, Artificial Sequence
  Amino acid sequence of LARD1
SIANLSTWVSHLPSAYGDGMTRVLESGFYEQMTRDSTIIVANLSDPARANTWVQDLNRNAEPHTGNTFIIGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 32 - protein, 140 aa, Artificial Sequence
  Amino acid sequence of LARD2
DSTIIVANLSDPARANTWVQDLNRNAEPHTGNTFIIGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 33 - protein, 10 aa, Artificial Sequence
  Acidic peptide consisting of 10 Asparatic acids
DDDDDDDDDD

SEQ ID NO: 34 - DNA, 32 bp, Artificial Sequence
  hlyBD-s primer
ggggagctcggattcttgtcataaaattgatt

SEQ ID NO: 35 - DNA, 31 bp, Artificial Sequence
  hlyBD-a primer
gggggatccttaacgctcatgtaaactttct

SEQ ID NO: 36 - protein, 458 aa, Artificial Sequence
  Negatively supercharged extensively (M37(-23))
MSRHHHHHHSYTKEQLMLAFSYMSYYGITHTGSAKENAELILEKMKEALETWEPFQEDDWEVVWGPAVYTMPFTIFNDAMMYVIQKEGAEGEYVIAIRGTNPVSISDWLFNDFMVSAMKEWPYASVEGEILKISESTSYGLKTLQELKPKSHIPGEDKTILEFLNEKIGPEGEAKICVTGHSKGGALSSTLALWLKDIQGVELSEDIDISTIPFAGPTAGNADFADYFDDCLGDQCTRIANSLDIVPYAWNTDSLEELKSIYISEEASVEPLLYQRALIEAMIAETEGKEYKQIKAETPPLEGDINPILIEYLVQAAYQHVVGYPELMGMMDDIPLTDIFEDAIAGLLELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 37 - protein, 458 aa, Artificial Sequence
  Negatively supercharged defensively (M37(-14))
MSRHHHHHHSYTKEQLMLAFSYMSYYGITHTGSAKKNAELILEKMKEALETWEPFQEDDWEVVWGPAVYTMPFTIFNDAMMYVIQKEGAEGEYVIAIRGTNPVSISDWLFNDFMVSAMKKWPYASVEGRILKISESTSYGLKTLQELKPKSHIPGEDKTILQFLNEKIGPEGEAKICVTGHSKGGALSSTLALWLKDIQGVELSQDIDISTIPFAGPTAGNADFADYFDDCLGDQCTRIANSLDIVPYAWNTNSLKKLKSIYISEQASVKPLLYQRALIRAMIAETKGKKYKQIKAETPPLEGNINPILIEYLVQAAYQHVVGYPELMGMMDDIPLTDIFEDAIAGLLELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 38 - protein, 458 aa, Artificial Sequence
  Selectively superneutralized (M37(Q))
MSRHHHHHHSYTKEQLMLAFSYMSYYGITHTGSAKQNAELILQKMKEALQTWKPFQEDDWEVVWGPAVYTMPFTIFNDAMMYVIQKQGAEGEYVIAIRGTNPVSISDWLFNDFMVSAMQQWPYASVEGRILQISESTSYGLKTLQQLQPQSHIPGENQTILQFLNEKIGPEGQAQICVTGHSKGGALSSTLALWLKDIQGVQLSQNIDISTIPFAGPTAGNADFADYFDDCLGDQCTRIANSLDIVPYAWNTNSLQQLKSIYISEQASVKPLLYQQALIQAMIAETQGQQYKQIKAETPPLEGNINPILIEYLVQAAYQHVVGYPELMGMMDDIPLTDIFEDAIAGLLELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 39 - protein, 458 aa, Artificial Sequence
  Randomly mutated and activity-based screened(M37(var))
MSRHHHHHHSYTKEQLMLAFSYMSYYGITHTGSAKKNAELILEKMKEALETWEPFQEDDWEVVWGPAVYTMPFTIFNDAMMYVIQKEGAEGEYVIAIRGTNPVSISDWLFNDFMVSAMKKWPYASVEGKVLKISESTSYGLKTLQELKPKSHIPGEDKTILQFLNEKIGPEGEAKICVTGHSKGGALSSTLALWLKDIQGVELSEDIDISTIPFAGPTAGNADFADYFDDCLGDQCTRIANSLDIVPYAWNTNSLKELKSIYISEEASVKPLLYQRALIEAMIAETEGKEYKQIKAETPPLEGNINPILIEYLVQAAYQHVVGYPELMGMMDDIPLTDIFEDAIAGLLELIEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS